(19) United States
(12) Patent Application Publication
(10) Pub. No.: US 2024/0293566 A1
(43) Pub. Date: Sep. 5, 2024

(54) ANTI-EGFRvIII ANTIBODY DRUG CONJUGATES AND USES THEREOF

(71) Applicant: Regeneron Pharmaceuticals, Inc., Tarrytown, NY (US)

(72) Inventors: Frank DELFINO, Poughquag, NY (US); Marcus KELLY, New York, NY (US); Jessica KIRSHNER, New York, NY (US); Thomas NITTOLI, Orangeburg, NY (US); Gavin THURSTON, Millerton, NY (US)

(21) Appl. No.: 18/572,930

(22) PCT Filed: Jun. 21, 2022
(86) PCT No.: PCT/US2022/034373

§ 371 Date: Dec. 21, 2023

Related U.S. Application Data
(60) Provisional application No. 63/213,478, filed on Jun. 22, 2021.
(60) Provisional application No. 63/242,929, filed on Sep. 10, 2021.

(51) Int. Cl. A61K 47/68; A61K 45/06; A61P 35/00

(52) U.S. Cl. CPC A61K 47/68035; A61K 45/06; A61K 47/6849; A61P 35/00

(57) ABSTRACT

The present disclosure provides antibody-drug conjugates (ADCs) comprising antibodies that bind to the class III variant of EGFR (EGFRVIII) conjugated to tesirine, and methods of using the same. According to certain embodiments, the antibodies or antigen-binding fragments thereof, useful herein, bind human EGFRVIII with high affinity. The antibodies or antigen-binding fragments thereof, useful herein, may be fully human antibodies. The ADCs provided herein are useful for the treatment of various cancers.

### CROSS REFERENCE TO RELATED APPLICATIONS

This application claims the benefit of U.S. Provisional Patent Application No. 63/213,478, filed Jun. 22, 2021; and U.S. Provisional Patent Application No. 63/242,929, filed Sep. 10, 2021; each of which is herein incorporated by reference in its entirety.

### FIELD OF THE DISCLOSURE

The present disclosure relates to antibody-drug conjugates (ADCs) comprising human antibodies and antigen-binding fragments of human antibodies that specifically bind the deletion mutants of human epidermal growth factor receptor (EGFR), in particular, the class III deletion mutant, EGFRvIII, where the antibody or antigen-binding fragment thereof is conjugated to tesirine, along with therapeutic methods of using those ADCs.

### SEQUENCE LISTING

An official copy of the sequence listing is submitted concurrently with the specification electronically via EFS-Web as an ASCII formatted sequence listing with a file name of 10966WO01_Sequence_Listing_ST25.TXT, a creation date of Jun. 21, 2022, and a size of about 49,152 bytes. The sequence listing contained in this ASCII formatted document is part of the specification and is herein incorporated by reference in its entirety.

### BACKGROUND

Overexpression and/or gene amplification of the epidermal growth factor (EGF) receptor, or EGFR, have been reported in multiple human tumors, including those in breast, ovarian, bladder, brain, and various squamous carcinomas (Wong, A. J. et al., 1987, Proc. Natl. Acad. Sci. USA, 84:6899-6903; Harris et al., 1992, Natl. Cancer Inst. Monogr. 11:181-187). However, targeting the EGFR as an anti-neoplastic therapeutic method has been problematic as many normal tissues also express this receptor and may get targeted along with the neoplastic targets. Meanwhile, it has been reported that many glioblastomas having EGFR gene amplification frequently contain gene rearrangement (Ekstrand, A. J. et al., 1992, Proc. Natl. Acad. Sci. USA, 89:4309-4313; Wong A. J. et al., 1992, Proc. Natl. Acad. Sci. USA, 89:2965-2969). In one study, 17 out of 44 glioblastomas were found to have one or more alterations in the EGFR coding sequence and all of these cases contained amplified EGFR, while none of the 22 cases without gene amplification showed any tumor-specific sequence abnormalities (Frederick, L. et al., 2000, Cancer Res 60:1383-1387). The same study also showed that multiple types of EGFR mutations could be detected in individual tumors.

The class Ill variant of the EGFR (EGFRvIII) is the most frequently found EGFR variant in glioblastoma (Bigner et al., 1990, Cancer Res 50:8017-8022; Humphrey et al., 1990, Proc Natl Acad Sci USA 87:4207-4211; Yamazaki et al., 1990, Jap J Cancer Res 81:773-779; Ekstrand et al., 1992, Proc Natl Acad Sci USA 89:4309-4313; Wikstrand et al., 1995, Cancer Res 55:3140-3148; and Frederick et al., 2000, Cancer Res 60:1383-1387). EGFRvIII is characterized by a deletion of exons 2-7 of the EGFR gene, resulting in an in-frame deletion of 801 base pairs of the coding region, i.e., deletion of 6-273 amino acid residues (based on the residue numbers of mature EGFR), as well as the generation of a new glycine at the fusion junction (Humphrey et al., 1988, Cancer Res 48:2231-2238; Yamazaki et al., 1990, supra). EGFRvIII has been shown to have a ligand-independent, weak but constitutively active kinase activity as well as enhanced tumorigenicity (Nishikawa et al., 1994, Proc Natl Acad Sci USA 91:7727-7731; and Batra et al., 1995, Cell Growth and Differentiation 6:1251-1259). In addition to gliomas, EGFRvIII has been detected in ductal and intraductal breast carcinoma (Wikstrand et al., 1995, Cancer Res 55:3140-3148), non-small cell lung carcinomas (Garcia de Palazzo et al., 1993, Cancer Res 53:3217-3220), ovarian carcinomas (Moscatello et al., 1995, Cancer Res 55:5536-5539), prostate cancer (Olapade-Olaopa et al., 2000, British J Cancer 82:186-194), and squamous cell carcinoma of the head and neck (Tinhofer et al., 2011, Clin Cancer Res 17(15):5197-5204). In contrast, these and other studies report that normal tissues do not express EGFRvIII (Garcia de Palazzo et al., 1993, supra; Wikstrand et al., 1995, supra; and Wikstrand et al., 1998, J Neuro Virol 4:148-158). The highly tumor-specific nature of EGFRvIII makes it an especially useful target for treating cancers and tumors that express this molecule.

The amino acid sequence of human EGFR is shown in SEQ ID NO: 27, and the amino acid sequence of EGFRvIII is shown in SEQ ID NO: 28. Antibodies to EGFRvIII are described in, for example, U.S. Pat. Nos. 5,212,290, 7,736,644, 7,589,180 and 7,767,792.

All publications, patent applications, patents and other references mentioned herein are incorporated by reference in their entirety.

### BRIEF SUMMARY OF THE DISCLOSURE

The present disclosure provides antibody-drug conjugates (ADCs) comprising antibodies and antigen-binding fragments thereof that bind EGFRvIII, wherein the antibodies and antigen-binding fragments thereof are conjugated to tesirine. Tesirine contains the pyrrolobenzodiazepine (PBD) payload/warhead, SG3199 (Tiberghien et al., 2016, ACS Medicinal Chemistry Letters 7(11):983-987). The ADCs are useful, inter alia, for targeting tumor cells that express EGFRvIII.

The antibodies useful in the ADCs provided herein can be full-length (for example, an IgG1 or IgG4 antibody) or may comprise only an antigen-binding portion (for example, a Fab, F(ab′)2 or scFv fragment), and may be modified to affect functionality, e.g., to eliminate residual effector functions (Reddy et al., 2000, J. Immunol. 164:1925-1933).

Exemplary anti-EGFRvIII antibodies useful herein are listed in Table 1. Table 1 sets forth the amino acid sequence identifiers of the heavy chain variable region (HCVR), light chain variable region (LCVR), heavy chain complementarity determining regions (HCDR1, HCDR2 and HCDR3), and light chain complementarity determining regions (LCDR1, LCDR2 and LCDR3) of an exemplary anti-EGFRvIII antibody. Table 2 sets forth full heavy and light chain amino acid sequences of an exemplary anti-EGFRvIII antibody. Table 3 sets forth the nucleic acid sequence identifiers of the HCVR, LCVR, HCDR1, HCDR2 HCDR3, LCDR1, LCDR2 and LCDR3 of an exemplary anti-EGFRvIII antibody.

The present disclosure provides ADCs comprising antibodies or antigen-binding fragments thereof that specifically bind EGFRvIII, comprising three complementarity determining regions (HCDR1, HCDR2, and HCDR3, respectively) within an HCVR comprising an amino acid sequence of SEQ ID NO: 2, or a substantially similar sequence thereof having at least 90%, at least 95%, at least 98% or at least 99% sequence identity thereto.

The present disclosure also provides ADCs comprising antibodies or antigen-binding fragments thereof that specifically bind EGFRvIII, comprising three complementarity determining regions (LCDR1, LCDR2, and LCDR3, respectively) within an LCVR comprising an amino acid sequence of SEQ ID NO: 10, or a substantially similar sequence thereof having at least 90%, at least 95%, at least 98% or at least 99% sequence identity thereto.

The present disclosure provides ADCs comprising antibodies or antigen-binding fragments thereof that specifically bind EGFRvIII, comprising an HCVR comprising an amino acid sequence of SEQ ID NO: 2, or a substantially similar sequence thereof having at least 90%, at least 95%, at least 98% or at least 99% sequence identity thereto.

The present disclosure also provides ADCs comprising antibodies or antigen-binding fragments thereof that specifically bind EGFRvIII, comprising an LCVR comprising an amino acid sequence of SEQ ID NO: 10, or a substantially similar sequence thereof having at least 90%, at least 95%, at least 98% or at least 99% sequence identity thereto.

The present disclosure also provides ADCs comprising antibodies or antigen-binding fragments thereof that specifically bind EGFRvIII, comprising an HCVR comprising an amino acid sequence of SEQ ID NO: 2 and an LCVR comprising an amino acid sequence of SEQ ID NO: 10.

The present disclosure also provides ADCs comprising antibodies or antigen-binding fragments thereof that specifically bind EGFRvIII, comprising a heavy chain CDR1 (HCDR1) comprising an amino acid sequence of SEQ ID NO: 4 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a heavy chain CDR2 (HCDR2) comprising an amino acid sequence of SEQ ID NO: 6 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a heavy chain CDR3 (HCDR3) comprising an amino acid sequence of SEQ ID NO: 8 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a light chain CDR1 (LCDR1) comprising an amino acid sequence of SEQ ID NO: 12 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a light chain CDR2 (LCDR2) comprising an amino acid sequence of SEQ ID NO: 14 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a light chain CDR3 (LCDR3) comprising an amino acid sequence of SEQ ID NO: 16 or a substantially similar sequence thereof having at least 90%, at least 95%, at least 98% or at least 99% sequence identity.

The present disclosure also provides ADCs comprising antibodies or antigen-binding fragments thereof that specifically bind EGFRvIII, comprising a set of six CDRs (i.e., HCDR1-HCDR2-HCDR3-LCDR1-LCDR2-LCDR3) contained within the HCVR of SEQ ID NO: 2 and the LCVR of SEQ ID NO: 10. In certain embodiments, the HCDR1-HCDR2-HCDR3-LCDR1-LCDR2-LCDR3 amino acid sequence set is: SEQ ID NO: 4, SEQ ID NO: 6; SEQ ID NO: 8; SEQ ID NO: 12; SEQ ID NO: 14; and SEQ ID NO: 16, respectively.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present disclosure includes ADCs comprising anti-EGFRvIII antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC). In some embodiments, an antibody or antigen-binding fragment thereof is aglycosylated. Aglycosylated antibodies are point mutated at a suitable residue to prevent glycosylation. In some aspects, the antibody or antigen-binding fragment thereof comprises a heavy chain which is aglycosylated at, for example, N297 (according to EU index numbering), to improve conjugation efficiency. In particular embodiments, the N297 is mutated to a glutamine (Q) residue, i.e., the antibody comprises an N297Q mutation.

In another aspect, the invention provides a complex comprising an anti-EGFRvIII-tesirine ADC, wherein the antibody or antigen-binding fragment thereof is bound to EGFRvIII.

In another aspect, the invention provides a pharmaceutical composition comprising an ADC comprising tesirine and a recombinant human antibody or fragment thereof which specifically binds EGFRvIII and a pharmaceutically acceptable carrier. In a related aspect, the invention features a composition which is a combination of an anti-EGFRvIII antibody-tesirine ADC and a second therapeutic agent. In one embodiment, the second therapeutic agent is any agent that is advantageously combined with an anti-EGFRvIII antibody-tesirine ADC. Exemplary combination therapies and co-formulations comprising the anti-EGFRvIII antibody-tesirine ADCs of the present disclosure are disclosed elsewhere herein.

In yet another aspect, the invention provides therapeutic methods for killing tumor cells or for inhibiting or attenuating tumor cell growth using an anti-EGFRvIII antibody-tesirine conjugate or antigen-binding portion of an antibody conjugated to tesirine. The therapeutic methods according to this aspect of the disclosure comprise administering a therapeutically effective amount of a pharmaceutical composition comprising an antibody-tesirine conjugate or antigen-binding fragment of an antibody conjugated to tesirine to a subject in need thereof. The disorder treated is any disease or condition which is improved, ameliorated, inhibited, or prevented by targeting the ADC to EGFRvIII.

Other embodiments will become apparent from a review of the ensuing detailed description. Other embodiments will become apparent from a review of the ensuing detailed description.

### BRIEF DESCRIPTION OF THE FIGURES

FIG. 1 shows a comparison of an anti-EGFRvIII-tesirine conjugate or anti-EGFRvIII-maytansinoid DM1 conjugate on tumor volume and weight 61 days post-implantation of 0.5×10⁶ MMT-EGFRvIII cells injected subcutaneously into the flank of female SCID mice.

### DETAILED DESCRIPTION

Before the present disclosure is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

For the purposes of describing and defining the present disclosure it is noted that the use of relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

In some examples, the term “substantially” in reference to a given parameter, property, or condition may mean and include to a degree that one of ordinary skill in the art would understand that the given parameter, property, or condition is met with a small degree of variance, such as within acceptable manufacturing tolerances. By way of example, depending on the particular parameter, property, or condition that is substantially met, the parameter, property, or condition may be at least 90% met, at least 95% met, at least 99% met, or fully met.

Although any methods and materials similar or equivalent to those described herein can be used in the practice or testing of the present disclosure, the exemplary methods and materials are now described. All patents, applications and non-patent publications mentioned in this specification are incorporated herein by reference in their entireties.

### Definitions

The term “EGFRvIII,” as used herein, refers to the human EGFR class Ill variant having the amino acid sequence shown in SEQ ID NO: 28, or a biologically active fragment thereof, which exhibits any characteristics specific for EGFRvIII, as opposed to those in common with normally expressed EGFR, unless specifically indicated otherwise. EGFRvIII lacks amino acid residues 6 through 273 of mature EGFR (i.e., SEQ ID NO: 27 without the signal peptide, i.e., residues 1-24) and contains a new glycine residue at position 6 between amino acid residues 5 and 274.

All references to proteins, polypeptides and protein fragments herein are intended to refer to the human version of the respective protein, polypeptide or protein fragment unless explicitly specified as being from a non-human species. Thus, the expression “EGFRvIII” means human EGFRvIII unless specified as being from a non-human species, e.g., “mouse EGFRvIII,” “monkey EGFRvIII,” etc.

As used herein, the expression “cell surface-expressed EGFRvIII” means one or more EGFRvIII protein(s), or the extracellular domain thereof, that is/are expressed on the surface of a cell in vitro or in vivo, such that at least a portion of a EGFRvIII protein is exposed to the extracellular side of the cell membrane and is accessible to an antigen-binding portion of an antibody. A “cell surface-expressed EGFRvIII” can comprise or consist of an EGFRvIII protein expressed on the surface of a cell which normally expresses EGFRvIII protein. Alternatively, “cell surface-expressed EGFRvIII” can comprise or consist of EGFRvIII protein expressed on the surface of a cell that normally does not express human EGFRvIII on its surface but has been artificially engineered to express EGFRvIII on its surface.

The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-EGFRvIII antibody (or antigen-binding portion thereof) may be identical to the human germline sequences, or may be naturally or artificially modified. An amino acid consensus sequence may be defined based on a side-by-side analysis of two or more CDRs.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies useful herein may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the disclosure in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments of the disclosure, the anti-EGFRvIII antibodies used herein are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies useful herein may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies useful herein may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-EGFRvIII antibodies useful herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes ADCs comprising antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies useful herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-EGFRvIII antibodies useful herein comprising variants of any of the HCVR, LCVR, and/or CDR amino acid sequences disclosed herein having one or more conservative substitutions. For example, the present disclosure includes anti-EGFRvIII antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a polypeptide, means that two peptide sequences, when optimally aligned, such as by the programs GAP or BESTFIT using default gap weights, share at least 95% sequence identity, even more preferably at least 98% or 99% sequence identity. In some aspects, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

A subject is a mammal, preferably a human.

### Anti-EGFRvIII Antibodies Comprising Fc Variants

According to certain embodiments of the present disclosure, anti-EGFRvIII antibodies useful herein comprise an Fc domain comprising one or more mutations which enhance or diminish antibody binding to the FcRn receptor, e.g., at acidic pH as compared to neutral pH. For example, the present disclosure includes ADCs comprising anti-EGFRvIII antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes ADCs comprising anti-EGFRvIII antibodies having an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations, and other mutations within the antibody variable domains disclosed herein, are contemplated within the scope of the present disclosure.

The present disclosure also includes ADCs comprising anti-EGFRvIII antibodies having a chimeric heavy chain constant (C_{H}) region, wherein the chimeric C_{H} region comprises segments derived from the C_{H} regions of more than one immunoglobulin isotype. For example, the antibodies useful herein may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies useful herein comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Provisional Appl. No. 61/759,578, filed Feb. 1, 2013, the disclosure of which is hereby incorporated by reference in its entirety).

In an embodiment of the invention, Fc is IgG4 having the mutation S108P.

### Antibody-Drug Conjugates (ADCs)

Provided herein are antibody-drug conjugates (ADCs) comprising an anti-EGFRvIII antibody or antigen-binding fragment thereof conjugated to tesirine.

Tesirine has the following structure:

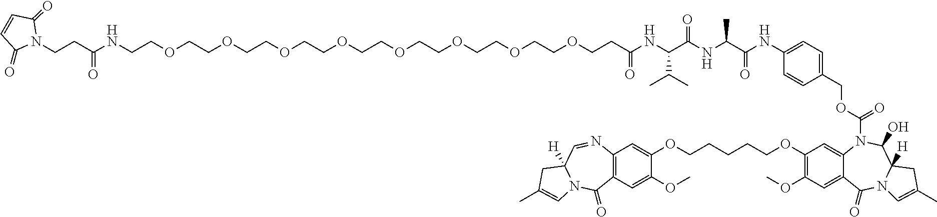 [SMILES: COc1cc2c(cc1OCCCCCOc1cc3c(cc1OC)C(=O)N1C=C(C)C[C@H]1[C@H](O)N3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=C[C@@H]1CC(C)=CN1C2=O]

Tesirine also referred to as SG3249.

Provided herein are compounds having the following structure:

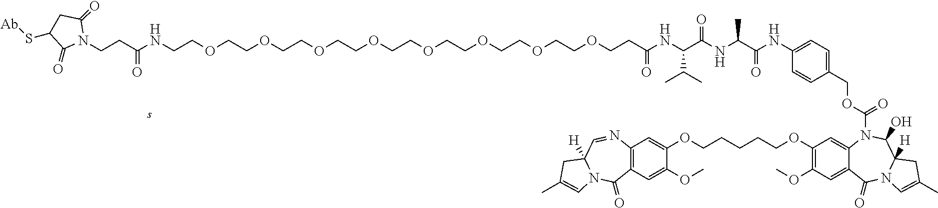

wherein Ab comprises an anti-EGFRvIII antibody or antigen-binding fragment thereof, and —S— is a sulfide bond at a cysteine residue of said antibody or antigen-binding fragment thereof. In particular embodiments, Ab comprises the three heavy chain CDRs within the HCVR amino acid sequence comprising SEQ ID NO: 2 and the three light chain CDRs within the LCVR amino acid sequence of SEQ ID NO: 10. In particular embodiments, Ab comprises an HCDR1 amino acid sequence of SEQ ID NO: 4, an HCDR2 amino acid sequence of SEQ ID NO: 6, an HCDR3 amino acid sequence of SEQ ID NO: 8, an LCDR1 amino acid sequence of SEQ ID NO: 12, an LCDR2 amino acid sequence of SEQ ID NO: 14, and an LCDR3 amino acid sequence of SEQ ID NO: 16. In particular embodiments, Ab comprises an HCVR amino acid sequence having at least 95%, at least 98%, or at least 99% sequence identity to the amino acid sequence of SEQ ID NO: 2 and an LCVR amino acid sequence having at least 95%, at least 98%, or at least 99% sequence identity to the amino acid sequence of SEQ ID NO: 10. In particular embodiments, Ab comprises an HCVR amino acid sequence of SEQ ID NO: 2 and/or an LCVR amino acid sequence of SEQ ID NO: 10.

Also provided herein are compounds having the following structure:

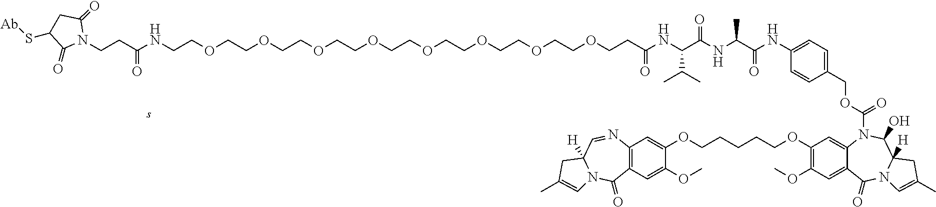

wherein Ab comprises an anti-EGFRvIII antibody or antigen-binding fragment thereof, and —S— is a sulfide bond at a cysteine residue of said antibody or antigen-binding fragment thereof. In particular embodiments, Ab is a full antibody. In particular embodiments, Ab comprises a heavy chain and a light chain, wherein the heavy chain comprises an amino acid sequence of SEQ ID NO: 18. In particular embodiments, Ab comprises a heavy chain and a light chain, wherein the heavy chain comprises an amino acid sequence of SEQ ID NO: 20. In particular embodiments, Ab comprises a heavy chain and a light chain, wherein the light chain comprises an amino acid sequence of SEQ ID NO: 22. In particular embodiments, Ab comprises a heavy chain comprising an amino acid sequence of SEQ ID NO: 18 and a light chain comprising an amino acid sequence of SEQ ID NO: 22. In particular embodiments, Ab comprises a heavy chain comprising an amino acid sequence of SEQ ID NO: 20 and a light chain comprising an amino acid sequence of SEQ ID NO: 22.

In some embodiments, the DAR (Drug-Antibody Ratio) is from about 1 to about 4. In some embodiments, the DAR is from about 2 to about 4. In some embodiments, the DAR is from about 2 to about 3. In some embodiments, the DAR is from about 3 to about 4. In some embodiments, the DAR is about 2. In some embodiments, the DAR is about 3. In some embodiments, the DAR is about 4.

The synthesis of tesirine can be performed, e.g., using procedures described in Tiberghien et al. (ACS Medicinal Chemistry Letters 2016, 7(11):983-987). Tesirine comprises the pyrrolobenzodiazepine warhead/payload component SG 3199, which has the following structure:

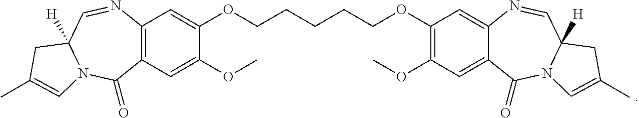

### Epitope Mapping and Related Technologies

The epitope to which the antibodies useful herein bind may consist of a single contiguous sequence of 3 or more (e.g., 3, 4, 5, 6, 7, 8, 9, 10, 11, 12, 13, 14, 15, 16, 17, 18, 19, 20 or more) amino acids of an EGFRvIII protein. Alternatively, the epitope may consist of a plurality of non-contiguous amino acids (or amino acid sequences) of EGFRvIII. In some embodiments, the epitope is located on or near the ligand-binding domain of EGFRvIII. In other embodiments, the epitope is located outside of the ligand-binding domain of EGFRvIII, e.g., at a location on the surface of EGFRvIII at which an antibody, when bound to such an epitope, does not interfere with ligand binding to EGFRvIII.

Antibodies and antigen-binding fragments thereof useful herein, according to certain embodiments, include anti-EGFRvIII antibodies that specifically bind EGFRvIII (and do not bind EGFR), wherein the antibodies recognize the EGFRvIII junctional peptide (e.g., SEQ ID NO:23). Such antibodies may be referred to herein as “junctional peptide binders,” “EGFRvIII peptide-binding antibodies,” and the like. According to other embodiments, anti-EGFRvIII antibodies useful herein specifically bind EGFRvIII (and do not bind EGFR), wherein the antibodies do not recognize the EGFRvIII junctional peptide (e.g. do not recognize the junctional peptide of SEQ ID NO:23, and/or do not recognize the peptide of SEQ ID NO:24). Such antibodies may be referred to herein as “conformational binders,” “EGFRvIII conformational epitope binders,” and the like.

Antibodies and antigen-binding fragments thereof, useful herein, include anti-EGFRvIII antibodies that bind to or interact with one or more residues within hEGFRvIII ECD(L25-A380).mmH (SEQ ID NO: 29), for example, bind to or interact with one or more residues corresponding to amino acids 64-82 GPCRKVCNGIGIGEFKDSL (SEQ ID NO: 26) of SEQ ID NO: 25 or SEQ ID NO: 29.

Various techniques known to persons of ordinary skill in the art can be used to determine whether an antibody or antigen-binding fragment thereof “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A.

The present disclosure further includes ADCs comprising anti-EGFRvIII antibodies that bind to the same epitope as any of the specific exemplary antibodies described herein (e.g. antibodies comprising any of the amino acid sequences as set forth in Table 1 herein). Likewise, the present disclosure also includes ADCs comprising anti-EGFRvIII antibodies that compete for binding to EGFRvIII with any of the specific exemplary antibodies described herein (e.g. antibodies comprising any of the amino acid sequences as set forth in Table 1 herein).

One can easily determine whether an antibody binds to the same epitope as, or competes for binding with, a reference anti-EGFRvIII antibody by using routine methods known in the art and exemplified herein. For example, to determine if a test antibody binds to the same epitope as a reference anti-EGFRvIII antibody of the disclosure, the reference antibody is allowed to bind to a EGFRvIII protein. Next, the ability of a test antibody to bind to the EGFRvIII molecule is assessed. If the test antibody is able to bind to EGFRvIII following saturation binding with the reference anti-EGFRvIII antibody, it can be concluded that the test antibody binds to a different epitope than the reference anti-EGFRvIII antibody. On the other hand, if the test antibody is not able to bind to the EGFRvIII molecule following saturation binding with the reference anti-EGFRvIII antibody, then the test antibody may bind to the same epitope as the epitope bound by the reference anti-EGFRvIII antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-EGFRvIII antibody, the above-described binding methodology is performed in two orientations: In a first orientation, the reference antibody is allowed to bind to an EGFRvIII protein under saturating conditions followed by assessment of binding of the test antibody to the EGFRvIII molecule. In a second orientation, the test antibody is allowed to bind to an EGFRvIII molecule under saturating conditions followed by assessment of binding of the reference antibody to the EGFRvIII molecule. If, in both orientations, only the first (saturating) antibody is capable of binding to the EGFRvIII molecule, then it is concluded that the test antibody and the reference antibody compete for binding to EGFRvIII. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Biological Characteristics of the Anti-EGFRvIII ADCs

The present invention includes anti-EGFRvIII-tesirine ADCs that bind specifically to EGFRvIII. In some aspects, the ADC comprises an anti-EGFRvIII antibody or antigen-binding fragment thereof binds neither: (i) the junctional peptide of SEQ ID NO: 23; nor (ii) the peptide of SEQ ID NO: 24. In some aspects, the ADC comprises an anti-EGFRvIII antibody or antigen-binding fragment thereof which exhibits an equilibrium dissociation constant (K_{D}) for a human EGFRvIII monomer of about 500 nM, as measured by a surface plasmon resonance assay at 37° C. In some aspects, the ADC comprises an anti-EGFRvIII antibody or antigen-binding fragment thereof which exhibits an equilibrium dissociation constant (K_{D}) for a human EGFRvIII dimer of about 10 nM or less, as measured by a surface plasmon resonance assay at 37° C. In some aspects, the ADC comprises an anti-EGFRvIII antibody or antigen-binding fragment thereof which does not bind an EGFR dimer at a level detectable by a surface plasmon resonance assay.

In some embodiments, the anti-EGFRvIII-tesirine ADC exhibits one or more of the following characteristics: (a) demonstrates reduced viability in vivo in EGFRvIII expressing cells; (b) demonstrates bystander cytotoxicity in vivo against non-EGFRvIII expressing cells co-cultured with EGFRvIII expressing cells; (c) demonstrates prolonged survival in mice with EGFRvIII expressing intracranial glioblastoma multiforme tumors; (d) demonstrates anti-tumor effect in mice with EGFRvIII expressing tumors in the absence of treatment related weight loss; (e) demonstrates tumor regression in mice with patient-derived glioblastoma multiforme tumors; (f) demonstrates greater tumor killing with lower dosages relative to a comparator antibody conjugated to MMAF; and/or (g) demonstrates greater anti-tumor potency than an anti-EGFRvIII-maytansinoid ADC in tumor bearing mice.

### Preparation of Human Antibodies

The anti-EGFRvIII antibodies or antigen-binding fragments thereof useful herein can be fully human antibodies. Methods for generating monoclonal antibodies, including fully human monoclonal antibodies are known in the art. Any such known methods can be used in the context of the present disclosure to make human antibodies that specifically bind to human EGFRvIII.

Using VELOCIMMUNE™ technology, for example, or any other similar known method for generating fully human monoclonal antibodies, high affinity chimeric antibodies to EGFRvIII are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-EGFRvIII antibody. While the constant region selected may vary according to specific use, high affinity antigen-binding and target specificity characteristics reside in the variable region. In certain instances, fully human anti-EGFRvIII antibodies are isolated directly from antigen-positive B cells.

The present invention includes a method for making an ADC that includes an antibody or antigen-binding fragment thereof of the present invention that specifically bind EGFRvIII comprising culturing a host cell comprising a polynucleotide that encodes an immunoglobulin that comprises the HCVR of said antibody or fragment and an immunoglobulin that comprises the LCVR of said antibody or fragment, in a culture medium, under conditions favorable to expression of the polynucleotide. One or more of the immunoglobulins of the antibody or fragment so produced can then be conjugated to tesirine, for example, by reducing (e.g., in the presence of dithiothreitol) the immunoglobulin chains and incubating said tesirine with the reduced immunoglobulin chains. A host cell in which such an antibody or fragment can be expressed is a eukaryotic or prokaryotic host cell, for example, a mammalian cell. Such host cells are well known in the art and many are available from the American Type Culture Collection (ATCC). These host cells include, inter alia, Chinese hamster ovary (CHO) cells, NSO, SP2 cells, Hela cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. ADCs produced by such a method for part of the present invention.

### Bioequivalents

The anti-EGFRvIII antibodies and antibody fragments useful herein encompass proteins having amino acid sequences that vary from those of the described antibodies but that retain the ability to bind human EGFRvIII. Such variant antibodies and antibody fragments comprise one or more additions, deletions, or substitutions of amino acids when compared to parent sequence, but exhibit biological activity that is essentially equivalent to that of the described antibodies. Likewise, the anti-EGFRvIII antibody-encoding DNA sequences of such antibodies encompass sequences that comprise one or more additions, deletions, or substitutions of nucleotides when compared to the disclosed sequence, but that encode an anti-EGFRvIII antibody or antibody fragment that is essentially bioequivalent to an anti-EGFRvIII antibody or antibody fragment of the disclosure. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-EGFRvIII antibodies useful herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-EGFRvIII antibody variants comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides anti-EGFRvIII antibodies useful herein that bind to human EGFRvIII but not to EGFRvIII from other species. The present disclosure also includes anti-EGFRvIII antibodies that bind to human EGFRvIII and to EGFRvIII from one or more non-human species. For example, the anti-EGFRvIII antibodies useful herein may bind to human EGFRvIII and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee EGFRvIII. According to certain exemplary embodiments, anti-EGFRvIII antibodies are provided which specifically bind human EGFRvIII and cynomolgus monkey (e.g., Macaca fascicularis) EGFRvIII. Other anti-EGFRvIII antibodies of the disclosure bind human EGFRvIII but do not bind, or bind only weakly, to cynomolgus monkey EGFRvIII.

### Therapeutic Formulation and Administration

The present disclosure provides pharmaceutical compositions comprising anti-EGFRvIII antibody-tesirine conjugates, i.e., an anti-EGFRvIII antibody-tesirine ADC. The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of ADC administered to a patient may vary depending upon the age and the size of the patient, target disease, conditions, route of administration, and the like. The preferred dose is typically calculated according to body weight or body surface area. In an adult patient, it may be advantageous to intravenously administer the antibody of the present disclosure normally at a single dose of about 0.001 to about 20 mg/kg body weight, more preferably about 0.002 to about 7, about 0.003 to about 5, or about 0.005 to about 3 mg/kg body weight. Exemplary dosages include 1 ug/kg, 3.5 ug/kg, 7 ug/kg, and 10 ug/kg. Depending on the severity of the condition, the frequency and the duration of the treatment can be adjusted. Effective dosages and schedules for administering anti-EGFRvIII antibody conjugates may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. As such, provided herein are methods for administering an anti-EGFRvIII antibody-tesirine ADC into the body of a subject, the method comprising injecting the ADC into the body of the subject. In some aspects, the ADC is injected into the body of the subject subcutaneously. In some aspects, the ADC is injected into the body of the subject intravenously. In some aspects, the ADC is injected into the body of the subject intramuscularly.

A pharmaceutical composition of the present disclosure can be provided in a vessel. A pharmaceutical composition of the present disclosure can be provided in an injection device. A pharmaceutical composition can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

Thus, the present invention includes methods for administering an ADC of the present invention, to a subject (e.g., wherein the subject suffers from cancer), including the steps of introducing the ADC into the body of the subject, e.g., by injection or any of the methods discussed herein.

The present invention also includes a vessel (e.g., glass or plastic vial; or a bag, such as an intravenous infusion bag) or any of such devices that include an ADC of the present invention, e.g., a syringe that includes a barrel, plunger and needle.

### Therapeutic Uses of the Anti-EGFRvIII Antibody Conjugates

The present disclosure includes methods comprising administering to a subject in need thereof a therapeutic composition comprising an antibody-drug conjugate comprising an anti-EGFRvIII antibody (e.g., an anti-EGFRvIII antibody or ADC comprising any of the HCVR/LCVR or CDR sequences as set forth in Table 1 herein) conjugated to tesirine. The therapeutic composition can comprise any of the anti-EGFRvIII antibodies, or antigen-binding fragments thereof, conjugated to tesirine, and a pharmaceutically acceptable carrier or diluent.

The ADCs of the disclosure are useful, inter alia, for the treatment, prevention and/or amelioration of any disease or disorder associated with or mediated by EGFRvIII expression or activity, or overexpression, or treatable by blocking the interaction between EGFRvIII and an EGFR ligand or otherwise inhibiting EGFRvIII activity and/or signaling, and/or promoting receptor internalization and/or decreasing cell surface receptor number. For example, the ADCs of the present disclosure are useful for the treatment of tumors that express EGFRvIII and/or that respond to ligand-mediated signaling. The ADCs of the present disclosure may also be used to treat primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the ADCs of the disclosure are used to treat one or more of the following cancers: glioblastoma, renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer (ductal or intraductal), or melanoma.

In the context of the methods of treatment described herein, the anti-EGFRvIII antibody-tesirine conjugate may be administered as a monotherapy (i.e., as the only therapeutic agent) or in combination with one or more additional therapeutic agents (examples of which are described elsewhere herein).

According to specific embodiments, the present disclosure provides methods for treating a cancer, reducing tumor growth and/or causing tumor regression in a patient. The methods according to this aspect of the disclosure comprise administering to a patient a first antibody-drug conjugate (ADC) either alone or in combination with a second anti-EGFRvIII antibody or ADC. The first ADC will typically comprise an antibody or antigen-binding fragment of an antibody and tesirine, wherein the antibody or antigen-binding fragment of the first ADC specifically binds EGFRvIII but does not bind the junctional EGFRvIII peptide of SEQ ID NO: 23 or the peptide of SEQ ID NO: 24 (i.e., the first ADC comprises a conformational EGFRvIII-binding antibody). In embodiments in which a second antibody or ADC is administered, the second antibody or ADC will typically comprise an antibody or antigen-binding fragment of an antibody and a cytotoxin, wherein the second antibody or antigen-binding fragment specifically binds EGFRvIII and also binds the junctional EGFRvIII peptide of SEQ ID NO: 23 and/or the peptide of SEQ ID NO: 24 (i.e., the second antibody or ADC comprises an EGFRvIII junctional peptide-binding antibody). When two separate anti-EGFRvIII ADCs are used in the context of this aspect of the disclosure, both ADCs may, in certain embodiments, comprise the same cytotoxic agent, i.e. both may comprise tesirine, or same class of cytotoxic agent. In other embodiments where two separate anti-EGFRvIII ADCs are used, each ADC may comprise a different cytotoxic agent and/or a different class of cytotoxic agent. According to certain embodiments, the antibody or antigen-binding fragment of the first ADC (i.e., the conformational EGFRvIII binding antibody) comprises heavy and light chain complementarity determining regions comprising SEQ ID NOs: 4, 6, 8, 12, 14, and 16, or the heavy chain variable region comprising SEQ ID NO: 2 and a light chain variable region comprising SEQ ID NO: 10.

### Combination Therapies and Formulations

The present disclosure includes compositions and therapeutic formulations comprising any of the anti-EGFRvIII antibody-tesirine conjugates described herein in combination with one or more additional therapeutically active components, and methods of treatment comprising administering such combinations to subjects in need thereof.

The anti-EGFRvIII antibody-tesirine conjugates useful herein may be co-formulated with and/or administered in combination with one or more additional therapeutically active component(s) selected from the group consisting of: a PRLR antagonist (e.g., an anti-PRLR antibody or small molecule inhibitor of PRLR), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2 [e.g., trastuzumab or T-DM1 {KADCYLA®}], anti-ErbB3 or anti-ErbB4 antibody or small molecule inhibitor of ErbB2, ErbB3 or ErbB4 activity), a cMET antagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody or small molecule kinase inhibitor such as, e.g., imatinib mesylate or sunitinib malate), a PDGF ligand inhibitor (e.g., anti-PDGF-A, -B, -C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 antagonist (e.g., an anti-FOLH1 antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin [e.g., anti-UPK3A] antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGR5 antagonist (e.g., an anti-LGR5 antibody), a monovalent CD20 antagonist (e.g., a monovalent anti-CD20 antibody such as rituximab), a PD-1 antibody, a PD-L1 antibody, a CD3 antibody, a CTLA-4 antibody etc. Other agents that may be beneficially administered in combination with the anti-EGFRvIII antibody-tesirine conjugates of the disclosure include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

The present disclosure includes compositions and therapeutic formulations comprising any of the anti-EGFRvIII antibody conjugates described herein in combination with one or more chemotherapeutic agents. Examples of chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

The anti-EGFRvIII antibody conjugates of the disclosure may also be administered and/or co-formulated in combination with antivirals, antibiotics, analgesics, corticosteroids, steroids, oxygen, antioxidants, COX inhibitors, cardioprotectants, metal chelators, IFN-gamma, and/or NSAIDs.

The additional therapeutically active component(s), e.g., any of the agents listed above or derivatives thereof, may be administered just prior to, concurrent with, or shortly after the administration of an anti-EGFRvIII antibody-tesirine conjugate of the present disclosure; (for purposes of the present disclosure, such administration regimens are considered the administration of an anti-EGFRvIII antibody-tesirine conjugate “in combination with” an additional therapeutically active component). The present disclosure includes pharmaceutical compositions in which an anti-EGFRvIII antibody-tesirine conjugate of the present disclosure is co-formulated with one or more of the additional therapeutically active component(s) as described elsewhere herein.

### Administration Regimens

According to certain embodiments of the present disclosure, multiple doses of an anti-EGFRvIII antibody-tesirine conjugate (or a pharmaceutical composition comprising a combination of an anti-EGFRvIII antibody-tesirine conjugate and any of the additional therapeutically active agents mentioned herein) may be administered to a subject over a defined time course. The methods according to this aspect of the disclosure comprise sequentially administering to a subject multiple doses of an anti-EGFRvIII antibody-tesirine conjugate of the disclosure. As used herein, “sequentially administering” means that each dose of anti-EGFRvIII antibody is administered to the subject at a different point in time, e.g., on different days separated by a predetermined interval (e.g., hours, days, weeks or months). The present disclosure includes methods which comprise sequentially administering to the patient a single initial dose of an anti-EGFRvIII antibody-tesirine conjugate, followed by one or more secondary doses of the anti-EGFRvIII antibody-tesirine conjugate, and optionally followed by one or more tertiary doses of the anti-EGFRvIII antibody-tesirine conjugate.

The terms “initial dose,” “secondary doses,” and “tertiary doses,” refer to the temporal sequence of administration of the anti-EGFRvIII antibody-tesirine conjugate of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-EGFRvIII antibody-tesirine conjugate, but generally may differ from one another in terms of frequency of administration. In certain embodiments, however, the amount of anti-EGFRvIII antibody-tesirine conjugate contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-EGFRvIII antibody-tesirine conjugate which is administered to a patient prior to the administration of the very next dose in the sequence with no intervening doses.

The methods according to this aspect of the disclosure may comprise administering to a patient any number of secondary and/or tertiary doses of an anti-EGFRvIII antibody-tesirine conjugate. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of once a month, then the maintenance doses may be administered to the patient once every six weeks, once every two months, once every three months, etc.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1. Generation of Anti-EGFRvIII Antibodies

Anti-EGFRvIII antibodies were obtained by immunizing a VELOCIMMUNE® mouse (i.e., an engineered mouse comprising DNA encoding human immunoglobulin heavy and kappa light chain variable regions) with an immunogen comprising the extracellular domain of EGFRvIII.

The antibody immune response was monitored by an EGFRvIII-specific immunoassay. When a desired immune response was achieved splenocytes were harvested and fused with mouse myeloma cells to preserve their viability and form hybridoma cell lines. The hybridoma cell lines were screened and selected to identify cell lines that produce EGFRvIII-specific antibodies. Using this technique, the exemplary H1H1863N2 anti-EGFRvIII chimeric antibody (i.e., possessing human variable domains and mouse constant domains) was obtained. The variable domain sequences for this antibody were initially disclosed in U.S. Pat. No. 9,475,875. This antibody is referred to herein as REGN1076. An aglycosylated version of the antibody, where the asparagine (N) at residue 297, as measured by EU index numbering, of the REGN1076 antibody heavy chain was mutated to a glutamine (Q) (i.e., H1H1863N2-N297Q), is referred to herein as REGN3124. The variable region sequences and the full heavy and light chain sequences are provided below.

Separately, REGN1076 with reduced fucosylation [“REGN1076(Fuc-)”] was prepared in a CHO host cell line that was described as “8088” in US Patent Application No. 2010/0304436A1, which is specifically incorporated by reference in its entirety. Mass spectrometry analysis of the resulting (Fuc-) antibody confirmed that core fucose was removed relative to the original antibody.

Table 1 sets forth the amino acid sequence identifiers of the heavy and light chain variable regions and CDRs of an exemplary anti-EGFRvIII antibody useful herein, while Table 2 provides the sequence identifiers for the full length heavy and light chain amino acid sequences. The corresponding nucleic acid sequence identifiers are set forth in Table 3.

TABLE 1
Sequence Identifiers for Variable Region Amino
Acid Sequences for REGN1076 and REGN3124
SEQ ID NOS:

| HCVR | HCDR1 | HCDR2 | HCDR3 | LCVR | LCDR1 | LCDR2 | LCDR3 |
|---|---|---|---|---|---|---|---|
| 2 | 4 | 6 | 8 | 10 | 12 | 14 | 16 |

TABLE 2
Sequence Identifiers for Full Heavy and Light Chain Amino Acid
Sequences for REGN1076 and REGN3124
SEQ ID NOs:

| Full length Heavy Chain | Full length Heavy Chain | Full length |
|---|---|---|
| (REGN1076) | with N297Q (REGN3124) | Light Chain |
| 18 | 20 | 22 |

TABLE 3
Sequence Identifiers for Variable Region Nucleic
Acid Sequences for REGN1076 and REGN3124
SEQ ID NOS:

| HCVR | HCDR1 | HCDR2 | HCDR3 | LCVR | LCDR1 | LCDR2 | LCDR3 |
|---|---|---|---|---|---|---|---|
| 1 | 3 | 5 | 7 | 9 | 11 | 13 | 15 |

As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1—will remain the same, and the binding properties are expected to be identical or substantially similar regardless of the nature of the Fc domain.

Antibodies were found to rapidly internalize into EGFRvIII positive tumor cells. Certain additional biological properties of the exemplary anti-EGFRvIII antibody generated in accordance with the methods of this Example are described in detail in the Examples set forth below.

### Control and Comparator Constructs Used in the Following Examples

Control constructs were included in the following experiments for comparative purposes: A comparator antibody, referred to herein as COMP, is a humanized anti-EGFRvIII antibody (hIgG1) with heavy and light chain variable domains having the amino acid sequences corresponding to SEQ ID NOS: 42 and 47, respectively, of the “hu806” antibody disclosed in U.S. Patent Application Publication No. 2010/0056762. The antibody is also referred to as ABT-414. The “hu806” antibody is known to bind to residues 311-326 (SEQ ID NO: 24) of EGFR (SEQ ID NO: 27), which is amplified or overexpressed, or residues 44-59 of EGFRvIII (SEQ ID NO: 28). COMP-MMAF refers to the ABT-414 antibody conjugated to monomethyl auristatin F (MMAF) via non-cleavable linker.

Control 1932 and Control 3892 are isotype control antibodies. Control 1932 has no Fc modifications and Control 3892 has an N297Q modification.

### Example 2. Tesirine-Antibody Conjugation and Characterization

Ten mg/mL each of the antibodies REGN1076 and REGN3124 and isotype control antibodies Control 1932 (no Fc modifications) and Control 3892 (having the N297Q modification) in 50 mM HEPES or PBS, 150 mM NaCl, pH 7.5, was treated with 1 mM dithiothreitol at 37° C. for 30 minutes. After gel filtration (G-25, pH 4.5 sodium acetate), the maleimido linker payload tesirine (aka, SG3249, synthesized as disclosed in Tiberghein et al., 2016, ACS Medicinal Chemistry Letters 7(11): 983-987) (1.2 equivalents/SH group) in DMSO (10 mg/mL) was added to the reduced antibody and the mixture adjusted to pH 7.0 with 1 M HEPES (pH 7.4). The conjugates were purified by size exclusion chromatography and sterile filtered. Protein concentrations were determined by UV and payload to antibody ratios were determined by mass spectrometry. Size-exclusion HPLC established that all conjugates used were >95% monomeric, and LC-MS established that there was <0.5% unconjugated linker payload. Payload to antibody ratios are shown in Table 4.

To determine the loading of tesirine on the antibody, the conjugates were deglycosylated, reduced, and analyzed by LC-MS.

For the assay, 50 ug of the conjugate was diluted with milli-Q water to a final concentration of 1 mg/mL. Ten μL of PNGase F solution [PNGase F solution was prepared by adding 150 μL of PNGase F stock (New England Biolabs, Cat #P0704L) and 850 μL of milli-Q water and mixed well] was added to the diluted conjugate solution and then incubated at 37° C. overnight. 2.4 μL of 0.5 M TCEP was added to the sample such that the resulting material had a final TCEP concentration of 20 mM and this was then incubated at 50° C. for 30 minutes. Injections of 10 μL of each sample were made onto LC-MS (Waters Synat G2-Si) and eluted with 0.1 mL/minute of a gradient mobile phase 20-40% over 25 minutes (Mobile Phase A: 0.1% v/v FA in H₂O; Mobile Phase B: 0.1% v/v FA in Acetonitrile). The LC separation was achieved on Waters Acquity BEH C18 column (1.0×50 mM, 1.7 μM).

The mass spectrometry spectra were deconvoluted and the identified light and heavy chain peaks represent the light chain (L) with linker-payload values=0 and 1, heavy chain (H) with linker-payload values=0, 1, 2, and 3. From the intensity values of each species, the drug to antibody ratio (DAR) was calculated using equation 1 below for a homo-dimer antibody conjugate. DARs for each conjugate are provided in Table 4.

$$DAR = 2 * [ \frac{L 1}{L 0 + L 1} + \frac{H 1 + 2 * H 2 + 3 * H 3}{H 0 + H 1 + H 2 + H 3} ] \qquad Equation 1$$

TABLE 4
Percent Yield and Payload to Antibody Ratios

| Antibody | Yield (%) | DAR (MS) |
|---|---|---|
| REGN1076-tesirine | 60 | 2.4-2.6 |
| REGN3124-tesirine | 80 | 3.4 |
| Control 3892-tesirine | 60 | 2.8 |

### Example 3. Biacore Binding Kinetics of EGFRvIII Monoclonal Antibodies

Equilibrium dissociation constants (K_{D} values) for EGFRvIII binding to PDB conjugates of anti-EGFRvIII antibodies were determined using a real-time surface plasmon resonance biosensor assay on a Biacore 2000 or 3000 instrument. The Biacore sensor surface was derivatized by amine coupling with a monoclonal mouse anti-human Fc antibody (GE Healthcare, #BR-1008-39) to capture the anti-EGFRvIII antibody drug conjugates and parent unmodified antibodies expressed with human constant regions. Biacore binding studies were performed in 0.01M HEPES pH 7.4, 0.15M NaCl, 3 mM EDTA, 0.05% v/v Surfactant P20 (HBS-EP running buffer). Different concentrations (3-fold dilutions) of human EGFRvIII extracellular domain expressed with a C-terminal myc-myc-hexahistidine tag (hEGFRvIII-MMH; SEQ ID NO: 29 (ranging from 600 nM to 22.2 nM) prepared in HBS-EP running buffer were injected over the anti-EGFRvIII antibody drug conjugate or antibody captured surface at a flow rate of 50 μL/minute. Association of hEGFRvIII-MMH to each of the captured antibody drug conjugates and monoclonal antibodies was monitored for 4 minutes. Subsequently, hEGFRvIII-MMH dissociation was monitored for 6-8 minutes in HBS-EP running buffer. The anti-human Fc surface was regenerated using an injection of 20 mM H₃PO₄. All binding kinetic experiments were performed at 25° C. Kinetic association (kₐ) and dissociation (k_{d}) rate constants were determined by fitting the real-time sensorgrams to a 1:1 binding model using Scrubber 2.0c curve fitting software. All sensorgrams were double referenced by subtracting buffer injection sensorgram signal from the corresponding analyte sensorgram, thereby removing artifacts caused by dissociation of the antibody from the capture surface. Binding dissociation equilibrium constants (K_{D}) and dissociative half-lives (t½) were calculated from the kinetic rate constants as:

$${K}_{D} ( M ) = {k}_{d} / {k}_{a} , and t 1 / 2 ( min ) = ln 2 / ( 60 \times {k}_{d} )$$

The binding kinetic parameters for hEGFRvIII-MMH binding to anti-EGFRvIII antibody drug conjugates and antibodies at 25° C. are shown in Table 5. As shown, the parental antibodies and their corresponding antibody drug conjugates demonstrated similar binding K_{D} values to the hEGFRvIII-MMH under the conditions tested.

TABLE 5
Biacore Kinetics of Human EGFRvIII-MMH Binding to Anti-
EGFRvIII Conjugates and Parent Unmodified Antibodies

| | mAb | Antigen | | | | |
|---|---|---|---|---|---|---|
| Parent mAb/ | Captured | Bound | kₐ | k_{d} | K_{D} | t1/2 |
| ADC Nr | (RU) | (RU) | (1/Ms) | (1/s) | (M) | (min) |
| REGN1076 | 398.4 ± 13.7 | 77 | 2.06E+04 | 2.69E−03 | 1.31E−07 | 4.3 |
| REGN1076- | 420.7 ± 17.7 | 117 | 2.23E+04 | 3.11E−03 | 1.40E−07 | 3.7 |
| tesirine | | | | | | |
| REGN3124 | 539.2 ± 47.5 | 167 | 1.89E+04 | 2.79E−03 | 1.48E−07 | 4.1 |
| REGN3124- | 420.9 ± 5.2 | 123 | 2.07E+04 | 2.67E−03 | 1.29E−07 | 4.3 |
| tesirine | | | | | | |

### Example 4. Cell Killing Activity of Anti-EGFRvIII Antibody-Tesirine ADCs

To determine the relative cell-killing potency of anti-EGFRvIII antibody drug conjugates of the invention, cell-killing assays were run on a cell line expressing human EGFRvIII. To develop the cell line, Lipofectamine LTX with Plus Reagent was used to generate U251 cells (Sigma, #9063001) expressing human EGFRvIII (hEGFRvIII; amino acids 1 through 380 of accession number NP_005219.2 with a deletion of amino acids 30 through 297 and creation of a junctional glycine residue, i.e. SEQ ID NO: 25) here in referred to as U251 MG/hEGFRvIII. The U251 lines were maintained in complete growth media (MEM Earle's Salts+10% FBS+1% L-glutamine/penicillin/streptomycin+1% non-essential amino acids+sodium pyruvate).

To measure the in vitro cytotoxicity of anti-EGFRvIII antibody drug conjugates, nuclear counts after a 6-day treatment with the antibody drug conjugates were assessed. Cells were seeded in 96 well plate (PerkinElmer, #6055308) at 3000 cells/well for U251 MG and U251/hEGFRvIII cells in complete growth media and grown overnight at 37° C. in 5% CO₂. For cell viability curves, serially diluted antibody drug conjugates and payload were added to the cells at final concentrations ranging from 100 nM to 1.5 pM (based on toxin concentration) and then incubated for 6 days at 37° C. in 5% CO₂. The last well in each dilution series (untreated wells) served as a blank control containing either the media alone (ADCs) or media plus 0.2% DMSO (payload) and was plotted as a continuation of the 3-fold serial dilution. Cells were subsequently treated with 3 ug/mL of Hoechst 33342 nuclear stain (ThermoFisher, #H3570) while being fixed with 4% formaldehyde (ThermoFisher, #28908), and images were acquired on the Opera Phenix (PerkinELmer). Nuclear counts were determined via Harmony image analysis software (PerkinELmer), and cell viability was expressed as a percentage of the untreated (100% viable) cells. IC₅₀ values were determined using a four-parameter logistic equation over a 10-point dose response curve (GraphPad Prism). The maximum % kill was also determined for each test article as follows: 100-minimum percent viability. The IC₅₀ value and maximum % kill of each test article is shown in Table 6.

As summarized in Table 6, anti-EGFRvIII antibody-drug conjugates REGN3124-tesirine and REGN1076-tesirine (glycosylated version of REGN3124) reduced cell viability, with IC₅₀ values of 33 pM for REGN3124-tesirine and 84 pM for REGN1076-tesirine in U251 MG/hEGFRvIII cells. REGN3124-tesirine and REGN1076-tesirine killed parental U251 MG cells with IC₅₀ values of 2.6 nM for REGN3124-tesirine and 4.9 nM for REGN1076-tesirine. The similarly conjugated isotype control antibody Control 3892-tesirine reduced cell viability with IC₅₀ values to 3.9 nM in U251 MG/hEGFRvIII cells and 1.8 nM in U251 MG parental cells. The free payload (SG3199) of tesirine killed U251 MG/hEGFRvIII cells with an IC₅₀ value of 10 pM and U251 MG parental cells with an IC₅₀ value of 2 pM.

REGN1076 conjugated to a comparator MMAF payload (REGN1076-MMAF) was also tested for cytotoxicity. Similar to the other tested anti-EGFRvIII ADCs, REGN1076-MMAF killed U251 MG/hEGFRvIII cells with 47 pM IC₅₀ values. The anti-EGFRVIII ADC REGN1076-MMAF was weakly cytotoxic in parental U251 MG cells with a 52 nM IC₅₀ value. The non-binding similarly conjugated isotype control antibody to MMAF (Control 1932-MMAF) was weakly cytotoxic in all tested lines with IC₅₀ greater than 100 nM.

TABLE 6
Cell Viability in U251/hEGFRvIII and Parental Cell Lines

| | | | | | | | U251MG/ | |
|---|---|---|---|---|---|---|---|---|
| | | | U251MG/ | | U251MG in | | hEGFRvlll in | |
| | U251MG | | hEGFRvIII | | coculture | | coculture | |
| Test | nM | % | nM | % | nM | % | nM | % |
| Articles | IC₅₀ | Kill | IC50 | Kill | IC₅₀ | Kill | IC₅₀ | Kill |
| REGN3124- | 2.6 | 90 | 0.033 | 90 | 0.028 | 91 | 0.043 | 88 |
| tesirine | | | | | | | | |
| REGN1076- | 4.9 | 90 | 0.084 | 88 | 0.059 | 92 | 0.082 | 86 |
| tesirine | | | | | | | | |
| Control | 1.8 | 91 | 3.9 | 86 | 2.4 | 92 | 4.0 | 84 |
| 3892-tesirine | | | | | | | | |
| SG3199 | 0.002 | 93 | 0.010 | 92 | 0.007 | 94 | 0.010 | 91 |
| REGN1076- | 52 | 84 | 0.047 | 87 | 28 | 79 | 0.015 | 87 |
| MMAF | | | | | | | | |
| Control | >100 | 11 | >100 | 14 | >100 | 0 | >100 | 75 |
| 1932-MMAF | | | | | | | | |

Bystander killing by an ADC can take place when the cytotoxic payload is released from the target cells and is then taken up by surrounding antigen-negative (bystander) cells. To assess potential bystander killing by REGN3124-tesirine and REGN1076-tesirine, U251 MG/hEGFRvIII cells were prelabeled with CellTrace™ Far red (Thermo Fisher, #C34564). A 1:1 coculture of 1500 cells/well of far red labeled U251 MG/hEGFRvIII cells and 1500 cells/well unlabeled U251 MG cells were incubated with either ADC or free payload M31 at a range of concentrations (100 nM to 1.5 pM) for 6 days. Cells were subsequently treated with 3 ug/mL of Hoechst 33342 nuclear stain (ThermoFisher, #H3570) while being fixed with 4% formaldehyde (ThermoFisher, #28908). Images were acquired on the Opera Phenix Microscope (PerkinELmer). All cells were identified by the Hoechst-labeled nuclei and cell counts were separated into far red positive U251 MG/hEGFRvIII cells (U251 in coculture) and far red negative parental U251 MG cell populations via Harmony image analysis software (PerkinELmer). Cell viability was determined separately for each cell population and expressed as a percentage of the untreated (100% viable) cells. IC₅₀ and maximum % kill values were determined as described previously and summarized in Table 6.

REGN3124-tesirine and REGN1076-tesirine killed U251 MG/hEGFRvIII cells from the coculture with IC₅₀ values of 43 pM and 82 pM, respectively, and killing was similar to that observed in U251 MG/hEGFRvIII mono-cultures. REGN3124-tesirine and REGN1076-tesirine also killed U251 MG parental cells from the coculture with IC₅₀ values of 28 pM and 59 pM, respectively, suggesting bystander killing activity by these ADCs. The non-binding ADC, Control 3892-tesirine killed U251 MG/hEGFRvIII and U251 MG parental cells with IC₅₀ values of 4.0 nM and 2.4 nM, respectively.

REGN1076 conjugated to a comparator MMAF payload (REGN1076-MMAF) was also tested for bystander activity. REGN1076-MMAF demonstrated potent cytotoxicity against U251 MG/hEGFRvIII cells from the cocultures with an IC₅₀ value of 15 pM. In contract to the tesirine conjugates, REGN1076-MMAF was weakly cytotoxic in U251 parental cells from the coculture with an IC₅₀ value of 28 nM. The non-binding ADC conjugated to MMAF (Control 1932-MMAF) was weakly cytotoxic in the coculture assay with IC₅₀ values>100 nM.

### Example 5. Hydrogen/Deuterium (H/D) Exchange Based Epitope Mapping of Anti-EGFRvIII Antibodies on Human Epidermal Growth Factor Receptor Variant (hEGFRvIII)

Hydrogen-Deuterium Exchange Mass Spectrometry (HDX-MS) was performed to determine the amino acid residues of Epidermal Growth Factor Receptor variant 3 (hEGFRvIII ECD(L25-A380).mmH (SEQ ID NO: 29), amino acid sequence in appendix) that interact with REGN3124. A general description of the HDX-MS method is set forth in e.g., Ehring (1999) Analytical Biochemistry 267(2):252-259; and Engen and Smith (2001) Anal. Chem. 73:256A-265A.

The HDX-MS experiments were performed on an integrated HDX/MS platform, consisting of a Leaptec HDX PAL system for the deuterium labeling and quenching, a Waters Acquity M-Class (Auxiliary solvent manager) for the sample digestion and loading, a Waters Acquity M-Class (μBinary solvent manager) for the analytical gradient, and Thermo Q Exactive HF mass spectrometer for peptide mass measurement.

The labeling solution was prepared as PBS buffer in D₂O at pD 7.0 (10 mM phosphate buffer, 140 mM NaCl, and 3 mM KCl, equivalent to pH 7.4 at 25° C.). For deuterium labeling, 10 μL of EGFRvIII (EGFRvIII extracellular domain (L25-A380) with a myc Histidine tag, SEQ ID NO: 29, 66 UM) or EGFRvIII premixed with REGN3124 in 1:0.6 molar ratio (Ag-Ab complex) was incubated at 20° C. with 90 μL D₂O labeling solution for various time-points (e.g., Undeuterated control=0 second; deuterium-labeled for 5 minutes, 20 minutes and 80 minutes). For each of the time-points, the experiment was performed in duplicates. The deuteration reaction was quenched by adding 100 μL of pre-chilled quench buffer (0.5 M TCEP-HCl, 8 M urea and 1% formic acid) to 100 μL of the sample. The mixed sample was incubated at 20° C. for 5 minutes. The quenched sample was then injected into a Waters HDX Manager for online pepsin/protease XIII digestion. The digested peptides were trapped onto a 1.0 mm×50 mm C8 column (NovaBioassays) and separated by a 13-minute gradient separation of 10%-32% B (mobile phase A: 0.5% formic acid in water, mobile phase B: 0.1% formic acid in acetonitrile). The separated peptides were analyzed by Q Exactive HF mass spectrometry in LC-MS/MS or LC-MS mode.

The LC-MS/MS data of undeuterated EGFRvIII sample were searched against a database including EGFRvIII and its randomized sequence using Byonic search engine (Protein Metrics) with default parameters for non-specific enzymatic digestion. A list of common human glycans is defined as potential variable modifications. The identified peptide list was then imported together with the LC-MS data from all deuterated samples into the HDX Workbench software (version 3.3) to calculate the deuterium uptake level of individual peptides in each replicate of the 3 HDX time-points.

For a given peptide, the centroid mass (intensity-weighted average mass) of the spectra are first calculated for the undeuterated (0 second) controls. The average centroid mass of the antigen and Ag-Ab complex undeuterated controls is considered as the mass for 0% percent deuterium incorporation (mass for 0% D). For each deuterated sample, absolute D-uptake is defined as the mass difference of the centroid mass of deuterated samples and mass for 0% D. Percent deuterium incorporation (% D) is determined by comparing the centroid mass to the masses for the 0 and 100% D (maximum D-uptake mass shift, defined as 80% of the mass difference between N-2 deuterium atoms and N-2 hydrogen atoms, where N equals the number of non-proline amino acids in the peptide).

$$Deuterium Uptake ( D - uptake ) = Average Mass ( deuterated ) - Average Mass ( undeuterated )$$

$$Percentage of deuterium uptake ( \% D ) = \frac{D - uptake for peptide at each time point \times 100 \%}{Maximum D - uptake of the peptide}$$

For each peptide, the absolute D-uptake and % D values were individually calculated for two replicates of each HDX time-point. For each HDX time-point, duplicate absolute D uptake and % D values were averaged for antigen and Ag-Ab complex. The mean of % D values of 5 min and 20 min HDX time-points is then presented as a single % D value for antigen or Ag-Ab complex, defined as ‘Antigen % D’ or ‘Ag-Ab % D’. The difference between Antigen % D and Ag-Ab % D is defined as delta % D (Δ%), representing the overall change in deuterium incorporation comparing antigen and Ag-Ab complex, for the given peptide.

A total of 200 peptides from hEGFRvIII were identified from both hEGFRvIII alone and hEGFRvIII in complex with REGN3124 samples, representing 84% sequence coverage of hEGFRvIII. Any peptide that exhibited greater than 5% decrease in percentage of deuterium uptake was defined as significantly protected (Δ% D<−5%). Peptides corresponding to amino acids 64-82 GPCRKVCNGIGIGEFKDSL (SEQ ID NO: 26) on hEGFRvIII were significantly protected by REGN3124.

TABLE 7
hEGFRvIII Peptides With Significant Protection Upon Formation
of hEGFRvIII-REGN3124 Complex Compared to hEGFRvIII Alone

| | 5 min | | | 20 min | | | |
|---|---|---|---|---|---|---|---|
| | | hEGFRvIII + | | | hEGFRvIII + | | |
| | hEGFRvIII | REGN3124 | | hEGFRvIII | REGN3124 | | Differential |
| hEGFRvIII.mmh | Centroid | Centroid | | Centroid | Centroid | | % D-uptake |
| Residues | MH⁺ | MH⁺ | ΔD | MH⁺ | MH⁺ | ΔD | Δ % D |
| 64-77 | 1411.39 | 1410.65 | −0.74 | 1411.75 | 1411.15 | −0.60 | −7 |
| 67-77 | 1152.40 | 1151.96 | −0.44 | 1152.75 | 1152.31 | −0.43 | −6 |
| 64-78 | 1559.57 | 1558.99 | −0.58 | 1559.92 | 1559.47 | −0.45 | −5 |
| 64-82 | 2005.03 | 2004.36 | −0.67 | 2005.44 | 2004.82 | −0.61 | −5 |

EGFRvIII ECD(L25-A380).mmH (mmH tag is underlined)
(SEQ ID NO: 29)
LEEKKGNYVVTDHGSCVRACGADSYEMEEDGVRKCKKCEGPCRKVCNGI
GIGEFKDSLSINATNIKHFKNCTSISGDLHILPVAFRGDSFTHTPPLDP
QELDILKTVKEITGFLLIQAWPENRTDLHAFENLEIIRGRTKQHGQFSL
AVVSLNITSLGLRSLKEISDGDVIISGNKNLCYANTINWKKLFGTSGQK
TKIISNRGENSCKATGQVCHALCSPEGCWGPEPRDCVSCRNVSRGRECV
DKCNLLEGEPREFVENSECIQCHPECLPQAMNITCTGRGPDNCIQCAHY
IDGPHCVKTCPAGVMGENNTLVWKYADAGHVCHLCHPNCTYGCTGPGLE
GCPTNGPKIPSIAEQKLISEEDLGGEQKLISEEDLHHHHHH

### Example 6. Anti-EGFRvIII Antibody-Tesirine ADCs Demonstrate Significant Anti-Tumor Efficacy Against EGFRvIII Transfected Glioblastoma Multiforme Cell Line Xenografts

The anti-tumor efficacy of REGN1076-tesirine and REGN3124-tesirine ADCs was initially assessed in glioblastoma cell line xenografts models transfected to express EGFRvIII as endogenous expression of the target is lost following in vitro culture. The first model assessed was U251/EGFRvIII, where tumors were established by the subcutaneous implantation of 10×10⁶ cells mixed 1:1 with Matrigel on the right flank of male SCID mice. Tumors were grown to ˜130 mm³ before treatment initiation, approximately 30 days post-implantation. Efficacy of the ADCs was also assessed in the U87/EGFRvIII where tumors were established by the subcutaneous implantation of 3×10⁶ cells on the right flank of male SCID mice. U87/EGFvIII tumors were grown to ˜190 mm³ before treatment initiation, approximately 25 days post-implantation. Mice were randomized into groups of 7-8 and treated with a single dose of test or control ADC. Tumor growth was monitored for 60-70 days post-treatment.

### Experimental Results:

An initial study in U251/EGFRvIII xenograft bearing mice assessed the activity of REGN1076-tesirine and REGN3124-tesirine anti-EGFRvIII ADCs following a single dose designed to deliver 2.5 or 5 ug/kg of PBD payload (Table 8). The growth of xenografts treated with Control-tesirine or Control-N297Q-tesirine ADC was not significantly delayed relative to vehicle control treated tumors. However, a significant delay in tumor growth was observed in tumors treated with REGN1076-tesirine or REGN3124-tesirine ADCs at the 2.5 ug/kg payload dose over the course of the study. The higher ADC doses that delivered 5 ug/kg of PBD payload had an even greater anti-tumor effect relative to control treatments. REGN3124-tesirine ADC produced a more durable anti-tumor effect relative to REGN1076-tesirine at equivalent dose levels. Overall, all anti-EGFRvIII treatment groups survived until completion of the study around 60 days post-dosing. No treatment related in weight and all groups were observed to gain approximately 10-15% of body weight over the course of the study.

Activity of REGN1076-tesirine ADC and REGN3124-tesirine ADC was also assessed in the U87/EGFRvIII tumor xenograft model (Table 9). Here a single dose of REGN1076-tesirine and REGN3124-tesirine ADCs was compared at a dose that delivered 2.5 ug/kg PBD payload. This model demonstrated very rapid growth and animals treated with vehicle control were euthanized 10 days post-dosing due to tumors reaching the study endpoint. Control-tesirine or Control-N297Q-tesirine ADCs mediated some delay of tumor growth although all tumors grew, and animals were euthanized at 24 days post dosing due to tumors reaching the study endpoint. Both REGN1076-tesirine and REGN3124-tesirine that delivered 2.5 ug/kg of PBD payload mediated significant and durable regression of tumor xenografts. All anti-EGFRvIII treated animals survived until completion of the study 70 days post-dosing. A single tumor in the REGN1076-tesirine demonstrated regrowth towards the end of the study. All tumors treated with REGN3124-tesirine remained suppressed. No treatment related in weight and all groups were observed to gain approximately 5% of body weight over the course of the study.

TABLE 8
Anti-EGFRvIII-Tesirine PBD ADCs mediated regression
of U251/EGFRvIII xenografts relative to controls
(Day 36 post-treatment).

| | | | | Tumor | |
|---|---|---|---|---|---|
| | | | | volume | |
| | | | | (mm³) at | Tumor growth |
| | | | | termination | (mm³) from |
| | | | | of vehicle | start of |
| | | ADC | Payload | group | treatment |
| Article | DAR | Dose | Dose | (mean ± SD) | (mean ± SD) |
| Vehicle | n/a | n/a | n/a | 1404 ± 490 | 1287 ± 485 |
| Control 1932- | 2.6 | 0.49 | 5 | 1051 ± 160 | 914 ± 139 |
| tesirine | | mg/kg | ug/kg | | |
| Control 3892- | 2.8 | 0.46 | 5 | 1130 ± 312 | 1012 ± 307 |
| tesirine | | mg/kg | ug/kg | | |
| REGN1076- | 2.4 | 0.27 | 2.5 | 419 ± 224 | 286 ± 174 |
| tesirine | | mg/kg | ug/kg | | |
| REGN1076- | 2.4 | 0.53 | 5 | 205 ± 55 | 73 ± 24 |
| tesirine | | mg/kg | ug/kg | | |
| REGN3124- | 3.4 | 0.19 | 2.5 | 339 ± 174 | 203 ± 151 |
| tesirine | | mg/kg | ug/kg | | |
| REGN3124- | 3.4 | 0.38 | 5 | 116 ± 66 | 0 ± 46 |
| tesirine | | mg/kg | ug/kg | | |

TABLE 9
Anti-EGFRvIII-Tesirine Conjugates Mediated
Regression of U87/EGFRvIII Xenografts Relative
to Controls (Day 10 Post-Treatment).

| | | | | Tumor | |
|---|---|---|---|---|---|
| | | | | volume | |
| | | | | (mm³) at | Tumor growth |
| | | | | termination | (mm³) from |
| | | | | of vehicle | start of |
| | | ADC | Payload | group | treatment |
| Article | DAR | Dose | Dose | (mean ± SD) | (mean ± SD) |
| Vehicle | n/a | n/a | n/a | 1284 ± 504 | 1095 ± 505 |
| Control 1932- | 2.6 | 0.25 | 2.5 | 479 ± 169 | 290 ± 160 |
| tesirine | | mg/kg | ug/kg | | |
| Control 3892- | 2.8 | 0.23 | 2.5 | 349 ± 80 | 164 ± 49 |
| tesirine | | mg/kg | ug/kg | | |
| REGN1076- | 2.4 | 0.27 | 2.5 | 154 ± 59 | −28 ± 43 |
| tesirine | | mg/kg | ug/kg | | |
| REGN3124- | 3.4 | 0.19 | 2.5 | 157 ± 35 | −33 ± 15 |
| tesirine | | mg/kg | ug/kg | | |

### Example 7. Anti-EGFRvIII Antibody-Tesirine ADCs Demonstrate Significant Anti-Tumor Efficacy Against Orthotopically Placed EGFRvIII Positive Glioblastoma Multiforme Patient Derived Xenografts

In order to assess the efficacy of anti-EGFRvIII ADCs against GBM tumors orthotopically placed in the brain, intracranial GBM6 (high and homogeneous EGFRvIII expression) or GBM59 (medium and heterogeneous EGFRvIII expression) patient derived xenograft (PDX) tumors were established by the injection of 3×10⁵ PDX cells. Intracranial injection was performed at 1 mm anterior and 2 mm lateral to the bregma at a depth of 3 mm. All orthotopic GBM PDX studies were conducted by Translational Drug Development Inc. Orthotopic GBM6 PDX were allowed to establish for 14+−1 days and GBM59 PDXs were allowed to establish for 25+−1 days before mice were randomized into groups of 7-8 and treated with a single dose of test or control ADC. Mice were monitored for ˜90 days for signs of peri-morbidity and euthanized prior to reaching a moribund state.

### Experimental Results:

In an initial study (Study A) with orthotopic GBM6 PDX tumor bearing mice, those treated with vehicle presented with rapidly deteriorating clinical signs and 7/8 mice were euthanized within 30 days of treatment (Table 10). Isotype control ADC did not result in any clinical effect and mice in this group were rapidly euthanized due to tumor induced per-morbidity. In contrast to the short 25 and 26.5 day median survivals observed in the control groups, treatment with REGN3124-tesirine (DAR 3.4) at 7 ug/kg payload dose resulted in a very significant prolongation of survival. The median survival in the anti-EGFRvIII-tesirine ADC group was not reached as 5/8 mice survived until the final observation point 94 days after dosing.

A second study (Study B) was initiated in mice bearing orthotopically placed GBM6 PDX tumors (Table 11). Again, isotype Control ADC mediated did not prolong survival relative to vehicle treated mice and the median survival of both groups was close to 20 days, with no survivors. REGN3124-tesirine (DAR 3.4) extended survival of at both the 3.5 and 7 ug/kg payload dose level, although the higher dose resulted in more mice (5/8) surviving until the completion of the study at Day 95 post treatment. REGN3124-tesirine (DAR 1.9) was similarly effective to REGN3124-tesirine (DAR 3.4), with a median survival of 77 days post treatment and 4/8 mice surviving until the end of the study. Rapid deterioration in animal body weight was observed in mice presenting with tumor-induced peri-morbidity. In contrast, animals treated with REGN3124-tesirine that demonstrated long term survival exhibited an associated 10-15% gain in body weight over the course of the post-treatment observation period.

The efficacy of REGN3124-tesirine was also assessed against orthotopically placed GBM59 PDX tumors (Table 12). In this study (Study C), all 8 mice treated with vehicle succumbed to the tumor burden with 30 days. Isotype control ADC mediated a partial prolongation of survival relative to vehicle control although all mice were euthanized due to peri-morbidity with 42 days post-treatment, resulting in a median survival of 32.5 days. As for the GBM6 model, very significant extension of survival was observed in mice receiving a single 7 ug/kg payload dose of REGN3124-tesirine. REGN3124-tesirine with DAR 1.9 and DAR 3.4 resulted in 7/8 mice surviving until completion of the study at Day 94 post treatment, and accordingly, the median survival in these groups was not reached. Less robust weight gain was observed in mice treated with DAR 1.9 REGN3124-tesirine relative to DAR 3.4 ADC in this study. Brains from the mice of Study C were taken at various timepoints, specifically when mice were euthanized due to evident disease, weight loss, or other clinical measures, or at completion of the study at Day 94 post treatment. Histological analysis was performed. No GBM59 cells were seen in any of the brains from mice treated with REGN3124-tesirine. Similar results were seen in GBM6 PDX studies (Study A). Subsequent immunohistochemistry demonstrated that the GMB59 PDX showed moderate and heterogeneous expression of EGFRvIII.

TABLE 10
Anti-EGFRvIII-Tesirine ADCs Significantly Prolonged the Survival
of Mice with Intracranial GBM6 GBM PDXs (Study A)

| | | | | N = 8 survival | P value |
|---|---|---|---|---|---|
| | | | | at Day 94 and | significance |
| | | ADC | Payload | median | from isotype |
| Article | DAR | Dose | Dose | survival (days) | control ADC |
| Vehicle | n/a | n/a | n/a | 1/8, MS = 25 | Not |
| | | | | days | significant |
| Control 3892- | 2.8 | 0.64 | 7 | 0/8, MS = 26.5 | Not |
| tesirine | | mg/kg | ug/kg | days | applicable |
| REGN3124- | 3.4 | 0.53 | 7 | 5/8, MS not | P < 0.0001 |
| tesirine | | mg/kg | ug/kg | reached | |

TABLE 11
Anti-EGFRvIII-Tesirine ADCs Significantly Prolonged the Survival
of Mice with Intracranial GBM6 GBM PDXs (Study B)

| | | | | N = 8 survival | P value |
|---|---|---|---|---|---|
| | | | | at Day 94 and | significance |
| | | ADC | Payload | median | from isotype |
| Article | DAR | Dose | Dose | survival (days) | control ADC |
| Vehicle | n/a | n/a | n/a | 0/8, MS = 20 | Not |
| | | | | days | significant |
| Control 3892- | 2.8 | 0.64 | 7 | 0/8, MS = 20.5 | Not |
| tesirine | | mg/kg | ug/kg | days | applicable |
| REGN3124- | 3.4 | 0.26 | 3.5 | 2/8, MS = 52 | P < 0.0001 |
| tesirine | | mg/kg | ug/kg | days | |
| REGN3124- | 3.4 | 0.53 | 7 | 5/8, MS not | P < 0.0001 |
| tesirine | | mg/kg | ug/kg | reached | |
| REGN3124- | 1.9 | 0.94 | 7 | 4/8, MS = 77 | P < 0.0001 |
| tesirine | | mg/kg | ug/kg | days | |

TABLE 12
Anti-EGFRvIII-Tesirine ADCs Significantly Prolonged the Survival
of Mice with Intracranial GBM59 GBM PDXs (Study C)

| | | | | N = 8 survival | P value |
|---|---|---|---|---|---|
| | | | | at Day 94 and | significance |
| | | ADC | Payload | median | from isotype |
| Article | DAR | Dose | Dose | survival (days) | control ADC |
| Vehicle | n/a | n/a | n/a | 0/8, MS = 17 | P = 0.0051 |
| | | | | days | |
| Control 3892- | 2.8 | 0.64 | 7 | 0/8, MS = 32.5 | Not |
| tesirine | | mg/kg | ug/kg | days | applicable |
| REGN3124- | 3.4 | 0.53 | 7 | 7/8, MS not | P < 0.0001 |
| tesirine | | mg/kg | ug/kg | reached | |
| REGN3124- | 1.9 | 0.94 | 7 | 7/8, MS not | P = 0.0009 |
| tesirine | | mg/kg | ug/kg | reached | |

### Example 8. REGN1076-Tesirine and REGN3124-Tesirine ADCs Demonstrate Significant Anti-Tumor Efficacy Against EGFRvIII Positive Glioblastoma Multiforme Patient Derived Xenografts

Patient derived xenografts with endogenous expression of EGFRvIII that represents the tumor biology of glioblastoma multiforme tumors were used to further examine the efficacy of REGN1076-tesirine and REGN3124-tesirine ADCs. GBM PDX studies were conducted by Translational Drug Development Inc. Subcutaneous tumors of GBM6 or GBM59 PDXs were established by the implantation of ˜50 mg of PDX fragments into the flank of nude mice. Once the tumor volumes reached approximately 125 mm³ 16-18 days post-implantation, mice were randomized into groups of 7-8 and treated with a single dose of test or control ADCs that delivered a dose equaling either 3.5 or 7 ug/kg pyrrolobenzodiazepine (PBD) payload dose. Tumor growth was monitored for 60 days post-treatment.

### Experimental Results:

In GBM6 PDX tumor bearing mice treated with vehicle, rapid tumor growth was observed, with tumors reaching the study endpoint 18 days post-treatment. Isotype control-tesirine ADCs only mediated a slight delay in tumor growth, with tumor reaching the study endpoint at Day 22 post-treatment. In contrast to vehicle and control treated tumors, anti-EGFRvIII ADCs mediated a very significant and durable tumor regression (Table 13). In the GBM6 model 3.5 ug/kg payload dose from REGN1076-tesirine or REGN3124-tesirine generally had equivalent anti-tumor efficacy, with 5/8 and 4/8 animals being tumor free at completion of the study on Day 60 post-treatment. REGN1076-tesirine or REGN3124-tesirine treatment that delivered 7 ug/kg PBD payload resulted in greater and more durable efficacy, with 7/8 and 8/8 being tumor free at completion of the study 60 days post-treatment. No treatment related weight loss was observed, with animal weights increasing by ˜15% over the course of the study.

TABLE 13
Anti-EGFRvIII-Tesirine ADCs Mediated Regression of GBM6
PDX Tumors Relative to Controls (Day 18 Post-Treatment).

| | | | | | Tumor volume | Tumor growth |
|---|---|---|---|---|---|---|
| | | | | | (mm³) at | (mm³) from |
| | | | | | termination of | start of |
| | | | | | vehicle group | treatment |
| Article | DAR | ADC Dose | Payload Dose | | (mean ± SD) | (mean ± SD) |
| Vehicle | n/a | n/a | n/a | | 2565 ± 843 | 2436 ± 809 |
| Control 1932- | 2.6 | 0.69 mg/kg | 7 | ug/kg | 1477 ± 573 | 1347 ± 534 |
| tesirine | | | | | | |
| Control 3892- | 2.8 | 0.64 mg/kg | 7 | ug/kg | 1707 ± 601 | 1577 ± 573 |
| tesirine | | | | | | |
| REGN1076- | 2.4 | 0.37 mg/kg | 3.5 | ug/kg | 65 ± 53 | −65 ± 51 |
| tesirine | | | | | | |
| REGN1076- | 2.4 | 0.75 mg/kg | 7 | ug/kg | 28 ± 18 | −102 ± 36 |
| tesirine | | | | | | |
| REGN3124- | 3.4 | 0.26 mg/kg | 3.5 | ug/kg | 83 ± 52 | −47 ± 41 |
| tesirine | | | | | | |
| REGN3124- | 3.4 | 0.53 mg/kg | 7 | ug/kg | 30 ± 11 | −100 ± 32 |
| tesirine | | | | | | |

The relative effect of REGN3124-tesirine ADCs with Drug:Antibody ratios (DARs) of 1.9 and 3.4 was assessed in GBM59 tumor bearing mice. Rapid tumor growth of mice treated with vehicle and control ADC was again observed, with both of these groups reaching the study endpoint 19 days post-treatment (Table 14). At the 3.5 ug/kg PBD dose, REGN3124-tesirine (DAR 3.4) mediated a moderate anti-tumor effect, and tumors reached the study endpoint at Day 30 post-treatment. REGN3124-tesirine (DAR 1.9) was also active, and tumors reached the study endpoint at Day 51 post-treatment with this agent. Consistent with other studies, REGN3124-tesirine ADC treatment that delivered 7 ug/kg payload dose resulted in greater and more durable inhibition of the GBM59 tumor growth. At this dose, REGN3124-tesirine (DAR 1.9) resulted in 3/7 tumors less than 50 mm³ at completion of the study on Day 60, whereas REGN3124-tesirine (DAR 3.4) resulted in 5/7 tumors less than 50 mm³ at study completion. No treatment related weight loss was observed, with animal weights increasing by ˜10% over the course of the study.

TABLE 14
Anti-EGFRvIII-Tesirine Conjugates Mediated Regression of GBM59
PDX Tumors Relative to Controls (Day 19 Post-Treatment).

| | | | | Tumor | |
|---|---|---|---|---|---|
| | | | | volume | |
| | | | | (mm³) at | Tumor growth |
| | | | | termination | (mm³) from |
| | | | | of vehicle | start of |
| | | ADC | Payload | group | treatment |
| Article | DAR | Dose | Dose | (mean ± SD) | (mean ± SD) |
| Vehicle | n/a | n/a | n/a | 2069 ± 788 | 1925 ± 750 |
| Control 3892- | 2.8 | 0.64 | 7 | 1910 ± 817 | 1753 ± 747 |
| tesirine | | mg/kg | ug/kg | | |
| REGN3124- | 3.4 | 0.26 | 3.5 | 851 ± 592 | 693 ± 534 |
| tesirine | | mg/kg | ug/kg | | |
| REGN3124- | 3.4 | 0.53 | 7 | 39 ± 39 | −118 ± 62 |
| tesirine | | mg/kg | ug/kg | | |
| REGN3124- | 1.9 | 0.47 | 3.5 | 585 ± 507 | 427 ± 453 |
| tesirine | | mg/kg | ug/kg | | |
| REGN3124- | 1.9 | 0.94 | 7 | 44 ± 39 | −114 ± 54 |
| tesirine | | mg/kg | ug/kg | | |

### Example 9. Assessment of Fractionated Dose Schedules of REGN3124-Tesirine ADC in EGFRvIII Positive GBM59 PDX Tumor Bearing Mice

A study was conducted using the subcutaneous GBM59 PDX tumor model to assess the effect of various dose schedules of REGN3124-tesirine conjugate on anti-tumor efficacy. This study was again conducted by Translational Drug Development Inc. Subcutaneous tumors GBM59 PDXs were established by the implantation of ˜50 mg of PDX fragments into the flank of nude mice. Once the tumor volumes reached approximately 125 mm³ 13 days post-implantation, mice were randomized into groups of 7 and treated with test or control ADCs. Isotype Control ADC was administered at a single dose equaling 7 ug/kg PBD payload. A low dose group of animals received ADC REGN3124-tesirine conjugate at 1.75 ug/kg per dose at Day 0 and 4 post treatment, resulting in a 3.5 ug/kg cumulative PBD dose. Further groups received REGN3124-tesirine delivering a cumulative 7 ug/kg dose. This was fractionated into individual doses of 3×2.33 ug/kg, 2×3.5 ug/kg or 1×7 ug/kg delivered on days 0, 4 and 8 (2.33 ug/kg) days 0 and 4 (3.5 ug/kg or day 0 (7 ug/kg). Tumor growth was monitored for 60 days post-treatment.

### Experimental Results:

In this study, Isotype Control ADCs did not cause any delay in tumor growth relative to vehicle control, and both groups were euthanized at Day 19 post treatment as mean tumor volume had reached the study endpoint (Table 15). In animals that received the 2×1.75 ug/kg REGN3124-tesirine, a significant inhibition of tumor volume was observed, and all mice survived until completion of the study 60 days post-treatment. All of the dose schedules that gave a cumulative PBD payload dose of 7 ug/kg resulted in a further and very significant anti-tumor effect. In mice dosed with REGN3124-tesirine at 3×2.33 ug/kg PBD dose, 3/7 were tumor free at the completion of the study and the mean tumor volume was below 90 mm³. In the groups that received REGN3124-tesirine that delivered 2×3.5 ug/kg and 1×7 ug/kg PBD dose, 2/7 and 3/7 mice were tumor free at study completion and the mean tumor volume for both groups was below 5 mm³, indicating the significant and durable efficacy of REGN3124-tesirine in this study. No treatment related weight loss was observed, with animal weights increasing by ˜10% over the course of the study.

TABLE 15
Anti-EGFRvIII-Tesirine Conjugates Mediated Regression of GBM6
PDX Tumors Relative to Controls (Day 18 post-treatment).

| | | | | Tumor volume | Tumor growth |
|---|---|---|---|---|---|
| | | | | (mm³) at | (mm³) from |
| | | | | termination of | start of |
| | | ADC | Payload | vehicle group | treatment |
| Article | DAR | Dose | Dose | (mean ± SD) | (mean ± SD) |
| Vehicle | n/a | n/a | n/a | 2457 ± 1359 | 2326 ± 1303 |
| Control 3892- | 2.8 | 1X 0.69 | 7 ug/kg | 2014 ± 1071 | 1880 ± 1062 |
| tesirine | | mg/kg | | | |
| REGN1076- | 1.9 | 2X 0.235 | 2X 1.75 ug/kg | 173 ± 46 | 40 ± 138 |
| tesirine | | mg/kg | (3.5 ug/kg) | | |
| REGN1076- | 1.9 | 3X 0.313 | 3X 2.33 ug/kg | 70 ± 84 | −65 ± 76 |
| tesirine | | mg/kg | (7 ug/kg) | | |
| REGN3124- | 1.9 | 2X 0.47 | 2X 3.5 ug/kg | 18 ± 13 | −114 ± 54 |
| tesirine | | mg/kg | (7 ug/kg) | | |
| REGN3124- | 1.9 | 1X 0.94 | 1X 7 ug/kg | 19 ± 12 | −116 ± 61 |
| tesirine | | mg/kg | | | |

### Example 10. Comparison of Anti-EGFRvIII-Tesirine ADC to Anti-EGFRvIII-Maytansinoid DM1 ADC

To establish tumors, 0.5×10⁶ MMT-EGFRvIII cells were injected subcutaneously into the flank of female SCID mice. Once the tumor volumes reached approximately 140 mm³ (Day 8), mice were randomized into groups of 7 and were treated with test and control ADCs using either the tesirine or DM1 payloads. Agents were dosed 3 days over a 17 day period. Tumor growth was monitored for 61 days post-implantation.

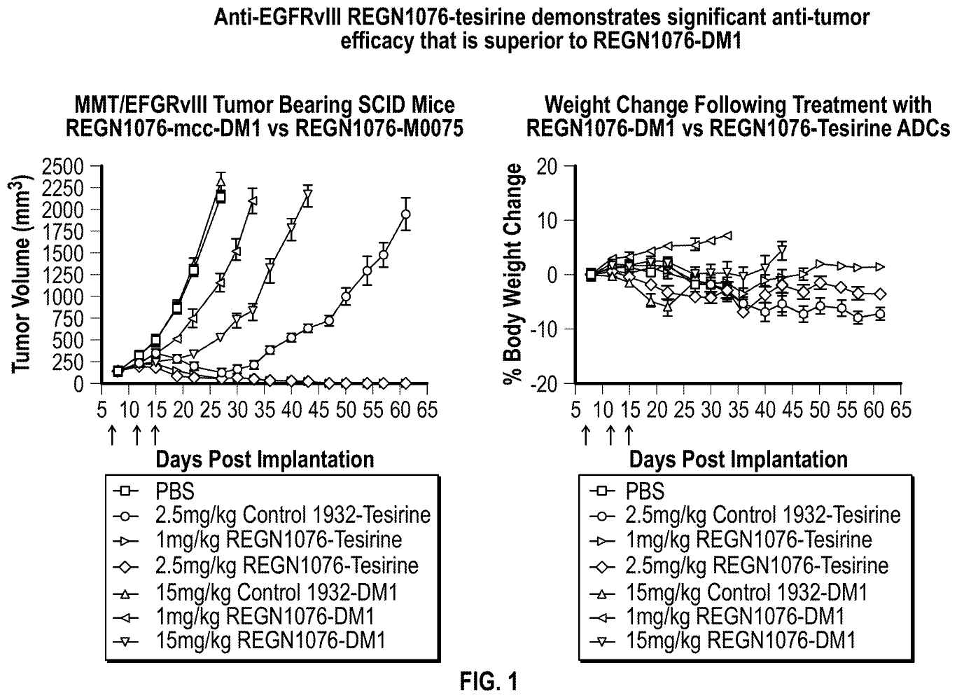

The anti-tumor efficacy of the respective EGFRvIII ADCs was then assessed over time (FIG. 1). In mice treated with 1 mg/kg REGN1076-tesirine ADC, complete tumor eradication was observed for the duration of the study. Control-tesirine ADC mediated a transient anti-tumor effect, although all tumors eventually demonstrated rapid progression towards the protocol endpoint tumor volume. In contrast to the significant efficacy observed following dosing with REGN1076-tesirine, REGN1076-DM1 administered at either 1 or 15 mg/kg only induced a moderate delay in tumor growth and all tumors rapidly progressed towards the protocol endpoint. Control DM1 ADC had no effect relative to vehicle control. The results of the anti-EGFRvIII at the 1 mg/kg dose level demonstrate the greater potency of anti-EGFRvIII-tesirine conjugate relative to the anti-EGFRvIII-maytansinoid DM1 conjugate. No treatment in this study induced severe weight loss.

### Example 11. Assessment of Anti-EGFRvIII-Tesirine Conjugate and COMP-MMAF ADC in EGFRvIII Positive Tumor Models

The activity of REGN3124-tesirine ADC was assessed simultaneously with that of a comparator ADC, COMP-MMAF (monomethyl auristatin F, an auristatin based ADC prepared based on the procedure described in Phillips et al., 2016, Mol Cancer Ther. 15(4): 661-669; see also Doronina et al., 2006, Bioconjugate Chem. 17:114-124), in the U251/EGFRvIII tumor xenograft model and in the intracranial orthotopic GBM59 PDX model. For the initial xenograft study, U251/EGFRvIII tumors were established by the subcutaneous implantation of 10×10⁶ cells mixed 1:1 with Matrigel on the right flank of male SCID mice. Tumors were grown to ˜175 mm³ before treatment initiation, approximately 30 days post-implantation. Mice were randomized into groups of 8 and treated with a single dose of test or control ADCs. Tumor growth was monitored for 71 days post-treatment.

In order to assess the efficacy of REGN3124-tesirine and the COMP-MMAF ADCs against GBM tumors orthotopically placed in the brain, intracranial GBM59 PDX tumors were established by the injection of 3×10⁵ PDX cells. Intracranial injection was performed at 1 mm anterior and 2 mm lateral to the bregma at a depth of 3 mm. All orthotopic GBM PDX studies were conducted by Translational Drug Development Inc. Orthotopic GBM59 PDX were allowed to establish for 25 days before mice were randomized into groups of 8 and treated with a single dose of test or control ADCs. Mice were monitored for 94 days for signs of peri-morbidity and euthanized prior to reaching a moribund state.

### Experimental Results:

A study in U251/EGFRvIII xenograft bearing mice assessed the activity of REGN3124-tesirine designed to deliver 7 ug/kg of PBD payload and the activity of the COMP-MMAF ADC at an ADC dose of 1, 2.5 and 5 mg/kg (Table 16). Isotype Controls were included for all dose levels in this study although only moderate anti-tumor effect was observed with these control agents. REGN3124-tesirine exhibited clear anti-tumor activity in this study, as the single dose of 0.53 mg/kg ADC (7 ug/kg PBD payload dose) was able to induce sustained regression of the xenografts. Sustained regression was not observed by tumors treated with 1 mg/kg COMP-MMAF. Activity against tumors was seen using 2.5 mg/kg COMP-MMAF, although 5 mg/kg COMP-MMAF was required to achieve sustained activity similar to that resulting from treatment with REGN3124-tesirine at completion of the study 71 days post-dosing. All animals in this study exhibited a 10% gain in body weight over the course of the post-treatment observation period.

TABLE 16
Anti-EGFRvIII-Tesirine Conjugate and COMP-MMAF Conjugate Mediated Regression
of U251/EGFRvIII Xenografts Relative to Controls (Day 36 Post-Treatment).

| | | | | | Tumor volume | Tumor growth |
|---|---|---|---|---|---|---|
| | | | | | (mm³) at | (mm³) from |
| | | | | | termination of | start of |
| | | | | Payload | vehicle group | treatment |
| Article | DAR | ADC Dose | | Dose | (mean ± SD) | (mean ± SD) |
| Vehicle | n/a | n/a | | n/a | 1811 ± 675 | 1636 ± 646 |
| Control 3892- | 2.8 | 0.64 | mg/kg | 7 ug/kg | 1112 ± 231 | 814 ± 379 |
| tesirine | | | | | | |
| REGN3124- | 3.4 | 0.53 | mg/kg | 7 ug/kg | 143 ± 63 | −24 ± 78 |
| tesirine | | | | | | |
| Control-MMAF | 3.0 | 1 | mg/kg | n/a | 1770 ± 347 | 1591 ± 338 |
| COMP-MMAF | 3.9 | 1 | mg/kg | n/a | 595 ± 325 | 423 ± 324 |
| Control-MMAF | 3.0 | 2.5 | mg/kg | n/a | 1556 ± 415 | 1383 ± 403 |
| COMP-MMAF | 3.9 | 2.5 | mg/kg | n/a | 183 ± 51 | 6 ± 36 |
| Control-MMAF | 3.0 | 5 | mg/kg | n/a | 1059 ± 380 | 881 ± 354 |
| COMP-MMAF | 3.9 | 5 | mg/kg | n/a | 136 ± 16 | −36 ± 22 |

The efficacy of REGN3124-tesirine (DAR 1.9) and the COMP-MMAF ADC was also assessed against orthotopically placed GBM59 PDX tumors (Table 17). In this study, all 8 mice treated with vehicle succumbed to the tumor burden with 25 days. Isotype Control-tesirine and Control MMAF ADCs did not prolong survival relative to the vehicle control group. In contrast to the control, REGN3124-tesirine mediated a very significant prolongation of survival, with 5/8 mice in this group surviving until completion of the study at Day 95 post-dosing. COMP-MMAF ADC at 1 mg/kg did not induce a significant increase in survival, as the median survival for this group was the same as Control-MMAF at 5 mg/kg. The higher 5 mg/kg COMP-MMAF treatment did induce some increase in survival relative to the MMAF control, although all mice succumbed to tumor burden within 35 days of treatment which resulted in a median survival of 23.5 days.

TABLE 17
Activity of REGN3124-Tesirine Conjugate
and COMP-MMAF Conjugate in Mice with Orthotopically
Placed GBM59 GBM PDX Tumors.

| | | | | N = 8 survival | P value |
|---|---|---|---|---|---|
| | | | | at Day 94 and | significance |
| | | ADC | Payload | median | from isotype |
| Article | DAR | Dose | Dose | survival (days) | control ADC |
| Vehicle | n/a | n/a | n/a | 0/8, MS = 15 | Not |
| | | | | days | significant |
| Control-tesirine | 2.9 | 0.62 | 7 | 0/8, MS = 24 | Not |
| | | mg/kg | ug/kg | days | applicable |
| REGN3124- | 1.9 | 0.95 | 7 | 5/8, MS not | P = 0.0002 |
| tesirine | | mg/kg | ug/kg | reached | |
| Control MMAF | 3.0 | 5 | n/a | 0/8, MS = 13.5 | Not |
| | | | | days | applicable |
| COMP-MMAF | 3.9 | 1 | n/a | 0/8, MS = 13.5 | Not |
| | | | | days | significant |
| COMP-MMAF | 3.9 | 5 | n/a | 0/8, MS = 23.5 | P = 0.016 |
| | | | | days | |

### Informal Sequence Listing

Provided below is an informal sequence listing reciting the sequences disclosed herein.

TABLE 18
Sequence Identifiers and Corresponding Nucleic Acid and Amino Acid
Sequences

| SEQ | | |
|---|---|---|
| ID | | |
| NO | Sequence | Description |
| 1 | gaggtgcagctggtggagtctgggggaggcttggtacagcctggggggtccctgagactctc | REGN1076 or REGN3124 |
| | ctgtgcagcctctggattccccttcagtagctacgacatgcactgggtccgccaagctacagg | HCVR nucleotide |
| | aaaaggtctggagtgggtctcagctattggtactgctggtgccacatactatccaggctccgtg | sequence |
| | aagggccgattcaccatctccagagaaaatgccaagaactccttgtatottcaaatgaacag | |
| | cctgagagccggggacacggctgtgtattactgtgcaagaggggattacgtttgggggactta | |
| | tcgtcccctctttgactactggggccagggaaccctggtcaccgtctcctca | |
| 2 | EVQLVESGGGLVQPGGSLRLSCAASGFPFSSYDMHWVRQATGK | REGN1076 or REGN3124 |
| | GLEWVSAIGTAGATYYPGSVKGRFTISRENAKNSLYLQMNSLRAG | HCVR amino acid |
| | DTAVYYCARGDYVWGTYRPLFDYWGQGTLVTVSS | sequence |
| 3 | gga ttc ccc ttc agt agc tac gac | REGN1076 or REGN3124 |
| | | HCDR1 nucleotide |
| | | sequence |
| 4 | G F P F S Y D | REGN1076 or REGN3124 |
| | | HCDR1 amino acid |
| | | sequence |
| 5 | att ggt act gct ggt gcc aca | REGN1076 or REGN3124 |
| | | HCDR2 nucleotide |
| | | sequence |
| 6 | I G T A G A T | REGN1076 or REGN3124 |
| | | HCDR2 amino acid |
| | | sequence |
| 7 | gca aga ggg gat tac gtt tgg ggg act tat cgt ccc ctc ttt gac tac | REGN1076 or REGN3124 |
| | | HCDR3 nucleotide |
| | | sequence |
| 8 | A R G D Y V W G T Y R P L F D Y | REGN1076 or REGN3124 |
| | | HCDR3 amino acid |
| | | sequence |
| 9 | gacatccagttgacccagtctccatccttcctgtctgcatctgtaggagacagagtcaccatca | REGN1076 or REGN3124 |
| | cttgctgggccagtcagggcattaacaattatttagcctggtatcaacaaaaaccagggaaag | LCVR nucleotide |
| | cccctaagctcctgatctatgctgcatccactttgcaaactggggtcccatcaaggttcagcgg | sequence |
| | cagtggatctgggacagaattcactctcacaatcagcagcctgcagcctgaagattttgcaac | |
| | ttattactgtcagcagcttaatagttacccgctcactttcggcggagggaccaaggtggagatc | |
| | aaa | |
| 10 | DIQLTQSPSFLSASVGDRVTITCWASQGINNYLAWYQQKPGKAPK | REGN1076 or REGN3124 |
| | LLIYAASTLQTGVPSRFSGSGSGTEFTLTISSLQPEDFATYYCQQL | LCVR amino acid |
| | NSYPLTFGGGTKVEIK | sequence |
| 11 | cag ggc att aac aat tat | REGN1076 or REGN3124 |
| | | LCDR1 nucleotide |
| | | sequence |
| 12 | Q G I N Y | REGN1076 or REGN3124 |
| | | LCDR1 amino acid |
| | | sequence |
| 13 | gct gca tcc | REGN1076 or REGN3124 |
| | | LCDR2 nucleotide |
| | | sequence |
| 14 | A A S | REGN1076 or REGN3124 |
| | | LCDR2 amino acid |
| | | sequence |
| 15 | cag cag ctt aat agt tac ccg ctc act | REGN1076 or REGN3124 |
| | | LCDR3 nucleotide |
| | | sequence |
| 16 | Q Q L N S Y P L T | REGN1076 or REGN3124 |
| | | LCDR3 amino acid |
| | | sequence |
| 17 | gaggtgcagctggtggagtctgggggaggcttggtacagcctggggggtccctgagactctc | REGN1076 heavy chain |
| | ctgtgcagcctctggattccccttcagtagctacgacatgcactgggtccgccaagctacagg | nucleotide sequence |
| | aaaaggtctggagtgggtctcagctattggtactgctggtgccacatactatccaggctccgtg | |
| | aagggccgattcaccatctccagagaaaatgccaagaactccttgtatcttcaaatgaacag | |
| | cctgagagccggggacacggctgtgtattactgtgcaagaggggattacgtttgggggactta | |
| | tcgtcccctctttgactactggggccagggaaccctggtcaccgtctcctcagcctccaccaag | |
| | ggcccatcggtcttccccctggcaccctcctccaagagcacctctgggggcacagcggccct | |
| | gggctgcctggtcaaggactacttccccgaaccggtgacggtgtcgtggaactcaggcgccc | |
| | tgaccagcggcgtgcacaccttcccggctgtcctacagtcctcaggactctactccctcagca | |
| | gcgtggtgaccgtgccctccagcagcttgggcacccagacctacatctgcaacgtgaatcac | |
| | aagcccagcaacaccaaggtggacaagaaagttgagcccaaatcttgtgacaaaactcac | |
| | acatgcccaccgtgcccagcacctgaactcctggggggaccgtcagtcttcctcttcccccca | |
| | aaacccaaggacaccctcatgatctcccggacccctgaggtcacatgcgtggtggtggacgt | |
| | gagccacgaagaccctgaggtcaagttcaactggtacgtggacggcgtggaggtgcataat | |
| | gccaagacaaagccgcgggaggagcagtacaacagcacgtaccgtgtggtcagcgtcctc | |
| | accgtcctgcaccaggactggctgaatggcaaggagtacaagtgcaaggtctccaacaaa | |
| | gccctcccagcccccatcgagaaaaccatctccaaagccaaagggcagccccgagaacc | |
| | acaggtgtacaccctgcccccatcccgggatgagctgaccaagaaccaggtcagcctgacc | |
| | tgcctggtcaaaggcttctatcccagcgacatcgccgtggagtgggagagcaatgggcagc | |
| | cggagaacaactacaagaccacgcctcccgtgctggactccgacggctccttcttcctctaca | |
| | gcaagctcaccgtggacaagagcaggtggcagcaggggaacgtcttctcatgctccgtgat | |
| | gcatgaggctctgcacaaccactacacgcagaagtccctctccctgtctccgggtaaatga | |
| 18 | EVQLVESGGGLVQPGGSLRLSCAASGFPFSSYDMHWVRQATGK | REGN1076 heavy chain |
| | GLEWVSAIGTAGATYYPGSVKGRFTISRENAKNSLYLQMNSLRAG | amino acid sequence |
| | DTAVYYCARGDYVWGTYRPLFDYWGQGTLVTVSSASTKGPSVFP | |
| | LAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTV | |
| | LHQDWLNGKEYKCKVSNKALPAPIEKTISKAKGQPREPQVYTLPP | |
| | SRDELTKNQVSLTCLVKGFYPSDIAVEWESNGQPENNYKTTPPVL | |
| | DSDGSFFLYSKLTVDKSRWQQGNVFSCSVMHEALHNHYTQKSLS | |
| | LSPGK* | |
| 19 | gaggtgcagctggtggagtctgggggaggcttggtacagcctggggggtccctgagactctc | REGN3124 heavy chain |
| | ctgtgcagcctctggattccccttcagtagctacgacatgcactgggtccgccaagctacagg | nucleotide sequence |
| | aaaaggtctggagtgggtctcagctattggtactgctggtgccacatactatccaggctccgtg | |
| | aagggccgattcaccatctccagagaaaatgccaagaactccttgtatcttcaaatgaacag | |
| | cctgagagccggggacacggctgtgtattactgtgcaagaggggattacgtttgggggactta | |
| | tcgtcccctctttgactactggggccagggaaccctggtcaccgtctcctcagcctccaccaag | |
| | ggcccatcggtcttccccctggcaccctcctccaagagcacctctgggggcacagcggccct | |
| | gggctgcctggtcaaggactacttccccgaaccggtgacggtgtcgtggaactcaggcgccc | |
| | tgaccagcggcgtgcacaccttcccggctgtcctacagtcctcaggactctactccctcagca | |
| | gcgtggtgaccgtgccctccagcagcttgggcacccagacctacatctgcaacgtgaatcac | |
| | aagcccagcaacaccaaggtggacaagaaagttgagcccaaatcttgtgacaaaactcac | |
| | acatgcccaccgtgcccagcacctgaactcctggggggaccgtcagtcttcctcttcccccca | |
| | aaacccaaggacaccctcatgatctcccggacccctgaggtcacatgcgtggtggtggacgt | |
| | gagccacgaagaccctgaggtcaagttcaactggtacgtggacggcgtggaggtgcataat | |
| | gccaagacaaagccgcgggaggagcagtaccaaagcacgtaccgtgtggtcagcgtcctc | |
| | accgtcctgcaccaggactggctgaatggcaaggagtacaagtgcaaggtctccaacaaa | |
| | gccctcccagcccccatcgagaaaaccatctccaaagccaaagggcagccccgagaacc | |
| | acaggtgtacaccctgcccccatcccgggatgagctgaccaagaaccaggtcagcctgacc | |
| | tgcctggtcaaaggcttctatcccagcgacatcgccgtggagtgggagagcaatgggcagc | |
| | cggagaacaactacaagaccacgcctcccgtgctggactccgacggctccttcttcctctaca | |
| | gcaagctcaccgtggacaagagcaggtggcagcaggggaacgtcttctcatgctccgtgat | |
| | gcatgaggctctgcacaaccactacacgcagaagtccctctccctgtctccgggtaaatga | |
| 20 | EVQLVESGGGLVQPGGSLRLSCAASGFPFSSYDMHWVRQATGK | REGN3124 heavy chain |
| | GLEWVSAIGTAGATYYPGSVKGRFTISRENAKNSLYLQMNSLRAG | amino acid sequence |
| | DTAVYYCARGDYVWGTYRPLFDYWGQGTLVTVSSASTKGPSVFP | |
| | LAPSSKSTSGGTAALGCLVKDYFPEPVTVSWNSGALTSGVHTFPA | |
| | VLQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVVV | |
| | DVSHEDPEVKFNWYVDGVEVHNAKTKPREEQYQSTYRVVSVLTV | |
| | LHQDWLNGKEYKCKVSNKALPAPIEKTISKAKGQPREPQVYTLPP | |
| | SRDELTKNQVSLTCLVKGFYPSDIAVEWESNGQPENNYKTTPPVL | |
| | DSDGSFFLYSKLTVDKSRWQQGNVFSCSVMHEALHNHYTQKSLS | |
| | LSPGK* | |
| 21 | gacatccagttgacccagtctccatccttcctgtctgcatctgtaggagacagagtcaccatca | REGN1076 and |
| | cttgctgggccagtcagggcattaacaattatttagcctggtatcaacaaaaaccagggaaag | REGN3124 light chain |
| | cccctaagctcctgatctatgctgcatccactttgcaaactggggtcccatcaaggttcagcgg | nucleotide sequence |
| | cagtggatctgggacagaattcactctcacaatcagcagcctgcagcctgaagattttgcaac | |
| | ttattactgtcagcagcttaatagttacccgctcactttcggcggagggaccaaggtggagatc | |
| | aaacgaactgtggctgcaccatctgtcttcatcttcccgccatctgatgagcagttgaaatctg | |
| | gaactgcctctgttgtgtgcctgctgaataacttctatcccagagaggccaaagtacagtggaa | |
| | ggtggataacgccctccaatcgggtaactcccaggagagtgtcacagagcaggacagcaag | |
| | gacagcacctacagcctcagcagcaccctgacgctgagcaaagcagactacgagaaaca | |
| | caaagtctacgcctgcgaagtcacccatcagggcctgagctcgcccgtcacaaagagcttc | |
| | aacaggggagagtgttag | |
| 22 | DIQLTQSPSFLSASVGDRVTITCWASQGINNYLAWYQQKPGKAPK | REGN1076 and |
| | LLIYAASTLQTGVPSRFSGSGSGTEFTLTISSLQPEDFATYYCQQL | REGN3124 light chain |
| | NSYPLTFGGGTKVEIKRTVAAPSVFIFPPSDEQLKSGTASVVCLLN | amino acid sequence |
| | NFYPREAKVQWKVDNALQSGNSQESVTEQDSKDSTYSLSSTLTL | |
| | SKADYEKHKVYACEVTHQGLSSPVTKSFNRGEC* | |
| 23 | LEEKKGNYVVTDH | Junctional Peptide |
| 24 | CGADSYEMEEDGVRKC | Other Peptide |
| 25 | LEEKKGNYVVTDHGSCVRACGADSYEMEEDGVRKCKKCEGPCR | Human EGFRvIII ECD |
| | KVCNGIGIGEFKDSLSINATNIKHFKNCTSISGDLHILPVAFRGDSFT | |
| | HTPPLDPQELDILKTVKEITGFLLIQAWPENRTDLHAFENLEIIRGRT | |
| | KQHGQFSLAVVSLNITSLGLRSLKEISDGDVIISGNKNLCYANTINW | |
| | KKLFGTSGQKTKIISNRGENSCKATGQVCHALCSPEGCWGPEPR | |
| | DCVSCRNVSRGRECVDKCNLLEGEPREFVENSECIQCHPECLPQ | |
| | AMNITCTGRGPDNCIQCAHYIDGPHCVKTCPAGVMGENNTLVWK | |
| | YADAGHVCHLCHPNCTYGCTGPGLEGCPTNGPKIPSIA | |
| 26 | GPCRKVCNGIGIGEFKDSL | Residues 64-82 of SEQ |
| | | ID NO: 25 |
| 27 | MRPSGTAGAALLALLAALCPASRALEEKKVCQGTSNKLTQLGTFE | Mature EGFR |
| | DHFLSLQRMFNNCEVVLGNLEITYVQRNYDLSFLKTIQEVAGYVLIA | |
| | LNTVERIPLENLQIIRGNMYYENSYALAVLSNYDANKTGLKELPMR | |
| | NLQEILHGAVRFSNNPALCNVESIQWRDIVSSDFLSNMSMDFQNH | |
| | LGSCQKCDPSCPNGSCWGAGEENCQKLTKIICAQQCSGRCRGKS | |
| | PSDCCHNQCAAGCTGPRESDCLVCRKFRDEATCKDTCPPLMLYN | |
| | PTTYQMDVNPEGKYSFGATCVKKCPRNYVVTDHGSCVRACGAD | |
| | SYEMEEDGVRKCKKCEGPCRKVCNGIGIGEFKDSLSINATNIKHFK | |
| | NCTSISGDLHILPVAFRGDSFTHTPPLDPQELDILKTVKEITGFLLIQ | |
| | AWPENRTDLHAFENLEIIRGRTKQHGQFSLAVVSLNITSLGLRSLK | |
| | EISDGDVIISGNKNLCYANTINWKKLFGTSGQKTKIISNRGENSCKA | |
| | TGQVCHALCSPEGCWGPEPRDCVSCRNVSRGRECVDKCNLLEG | |
| | EPREFVENSECIQCHPECLPQAMNITCTGRGPDNCIQCAHYIDGP | |
| | HCVKTCPAGVMGENNTLVWKYADAGHVCHLCHPNCTYGCTGPG | |
| | LEGCPTNGPKIPSIATGMVGALLLLLVVALGIGLFMRRRHIVRKRTL | |
| | RRLLQERELVEPLTPSGEAPNQALLRILKETEFKKIKVLGSGAFGTV | |
| | YKGLWIPEGEKVKIPVAIKELREATSPKANKEILDEAYVMASVDNPH | |
| | VCRLLGICLTSTVQLITQLMPFGCLLDYVREHKDNIGSQYLLNWCV | |
| | QIAKGMNYLEDRRLVHRDLAARNVLVKTPQHVKITDFGLAKLLGAE | |
| | EKEYHAEGGKVPIKWMALESILHRIYTHQSDVWSYGVTVWELMTF | |
| | GSKPYDGIPASEISSILEKGERLPQPPICTIDVYMIMVKCWMIDADS | |
| | RPKFRELIIEFSKMARDPQRYLVIQGDERMHLPSPTDSNFYRALMD | |
| | EEDMDDVVDADEYLIPQQGFFSSPSTSRTPLLSSLSATSNNSTVA | |
| | CIDRNGLQSCPIKEDSFLQRYSSDPTGALTEDSIDDTFLPVPEYINQ | |
| | SVPKRPAGSVQNPVYHNQPLNPAPSRDPHYQDPHSTAVGNPEYL | |
| | NTVQPTCVNSTFDSPAHWAQKGSHQISLDNPDYQQDFFPKEAKP | |
| | NGIFKGSTAENAEYLRVAPQSSEFIGA | |
| 28 | MRPSGTAGAALLALLAALCPASRALEEKKGNYVVTDHGSCVRAC | hEGFR class 3 variant |
| | GADSYEMEEDGVRKCKKCEGPCRKVCNGIGIGEFKDSLSINATNIK | |
| | HFKNCTSISGDLHILPVAFRGDSFTHTPPLDPQELDILKTVKEITGFL | |
| | LIQAWPENRTDLHAFENLEIIRGRTKQHGQFSLAVVSLNITSLGLRS | |
| | LKEISDGDVIISGNKNLCYANTINWKKLFGTSGQKTKIISNRGENSC | |
| | KATGQVCHALCSPEGCWGPEPRDCVSCRNVSRGRECVDKCNLL | |
| | EGEPREFVENSECIQCHPECLPQAMNITCTGRGPDNCIQCAHYID | |
| | GPHCVKTCPAGVMGENNTLVWKYADAGHVCHLCHPNCTYGCTG | |
| | PGLEGCPTNGPKIPSIATGMVGALLLLLVVALGIGLFMRRRHIVRKR | |
| | TLRRLLQERELVEPLTPSGEAPNQALLRILKETEFKKIKVLGSGAFG | |
| | TVYKGLWIPEGEKVKIPVAIKELREATSPKANKEILDEAYVMASVDN | |
| | PHVCRLLGICLTSTVQLITQLMPFGCLLDYVREHKDNIGSQYLLNW | |
| | CVQIAKGMNYLEDRRLVHRDLAARNVLVKTPQHVKITDFGLAKLLG | |
| | AEEKEYHAEGGKVPIKWMALESILHRIYTHQSDVWSYGVTVWELM | |
| | TFGSKPYDGIPASEISSILEKGERLPQPPICTIDVYMIMVKCWMIDA | |
| | DSRPKFRELIIEFSKMARDPQRYLVIQGDERMHLPSPTDSNFYRAL | |
| | MDEEDMDDVVDADEYLIPQQGFFSSPSTSRTPLLSSLSATSNNST | |
| | VACIDRNGLQSCPIKEDSFLQRYSSDPTGALTEDSIDDTFLPVPEYI | |
| | NQSVPKRPAGSVQNPVYHNQPLNPAPSRDPHYQDPHSTAVGNP | |
| | EYLNTVQPTCVNSTFDSPAHWAQKGSHQISLDNPDYQQDFFPKE | |
| | AKPNGIFKGSTAENAEYLRVAPQSSEFIGA | |
| 29 | LEEKKGNYVVTDHGSCVRACGADSYEMEEDGVRKCKKCEGPCR | human EGFRvIII |
| | KVCNGIGIGEFKDSLSINATNIKHFKNCTSISGDLHILPVAFRGDSFT | extracellular domain |
| | HTPPLDPQELDILKTVKEITGFLLIQAWPENRTDLHAFENLEIIRGRT | expressed with a C- |
| | KQHGQFSLAVVSLNITSLGLRSLKEISDGDVIISGNKNLCYANTINW | terminal myc-myc- |
| | KKLFGTSGQKTKIISNRGENSCKATGQVCHALCSPEGCWGPEPR | hexahistidine tag |
| | DCVSCRNVSRGRECVDKCNLLEGEPREFVENSECIQCHPECLPQ | |
| | AMNITCTGRGPDNCIQCAHYIDGPHCVKTCPAGVMGENNTLVWK | |
| | YADAGHVCHLCHPNCTYGCTGPGLEGCPTNGPKIPSIAEQKLISEE | |
| | DLGGEQKLISEEDLHHHHHH | |

The present disclosure is not to be limited in scope by the specific embodiments described herein. Indeed, various modifications of the disclosure in addition to those described herein will become apparent to those skilled in the art from the foregoing description and the accompanying FIGURES. Such modifications are intended to fall within the scope of the appended claims.

What is claimed is:

1. An antibody-drug conjugate (ADC) comprising an antibody or antigen-binding fragment thereof that binds specifically to EGFRvIII, wherein the antibody or antigen-binding fragment thereof comprises:
a heavy chain variable region (HCVR) comprising three heavy chain complementarity determining regions (HCDR1, HCDR2 and HCDR3) within a heavy chain variable region (HCVR) that comprises the amino acid sequence of SEQ ID NO: 2; and
a light chain variable region (LCVR) comprising three light chain complementarity determining regions (LCDR1, LCDR2 and LCDR3) within a light chain variable region (LCVR) that comprises the amino acid sequence of SEQ ID NO: 10;
and wherein the antibody is conjugated to tesirine.

2. The ADC of claim 1, wherein the anti-EGFRvIII antibody or antigen-binding fragment thereof binds neither:
(i) the junctional peptide of SEQ ID NO: 23; nor
(ii) the peptide of SEQ ID NO: 24.

3. The ADC of claim 1, wherein the antibody or antigen-binding fragment thereof:
(a) exhibits an equilibrium dissociation constant (K_{D}) for a human EGFRvIII monomer of about 500 nM, as measured by a surface plasmon resonance assay at 37° C.;
(b) exhibits an equilibrium dissociation constant (K_{D}) for a human EGFRvIII dimer of about 10 nM or less, as measured by a surface plasmon resonance assay at 37° C.; or
(c) does not bind an EGFR dimer at a level detectable by a surface plasmon resonance assay.

4. (canceled)

5. (canceled)

6. The ADC of claim 1, wherein the antibody or antigen-binding fragment thereof comprises:
an HCVR that comprises,
an HCDR1 that comprises the amino acid sequence of SEQ ID NO: 4,
an HCDR2 that comprises the amino acid sequence of SEQ ID NO: 6, and
an HCDR3 that comprises the amino acid sequence of SEQ ID NO: 8,
and an LCVR that comprises,
an LCDR1 that comprises the amino acid sequence of SEQ ID NO: 12,
an LCDR2 that comprises the amino acid sequence of SEQ ID NO: 14, and
an LCDR3 that comprises the amino acid sequence of SEQ ID NO: 16.

7. (canceled)

8. The ADC of claim 1, wherein the antibody or antigen-binding fragment thereof comprises:
an HCVR that comprises an amino acid sequence having at least 98% sequence identity to the amino acid sequence of SEQ ID NO: 2; and
an LCVR that comprises an amino acid sequence having at least 98% sequence identity to the amino acid sequence of SEQ ID NO: 10.

9. (canceled)

10. The ADC of claim 1, wherein the antibody or antigen-binding fragment thereof comprises:
an HCVR that comprises the amino acid sequence of SEQ ID NO: 2; and
an LCVR that comprises the amino acid sequence of SEQ ID NO: 10.

11. The ADC of claim 1, wherein the antibody or antigen-binding fragment thereof is a full antibody.

12. The ADC of claim 1, wherein the antibody or antigen-binding fragment thereof comprises a heavy chain and a light chain, wherein:
(a) the heavy chain comprises an amino acid sequence of SEQ ID NO: 18 or SEQ ID NO: 20; and
(b) the light chain comprises an amino acid sequence of SEQ ID NO: 22.

13. (canceled)

14. The ADC of claim 1, wherein the antibody or antigen-binding fragment thereof comprises:
(a) a heavy chain comprising an amino acid sequence of SEQ ID NO: 18 and a light chain comprising an amino acid sequence of SEQ ID NO: 22; or
(b) a heavy chain comprising an amino acid sequence of SEQ ID NO: 20 and a light chain comprising an amino acid sequence of SEQ ID NO: 22.

15. (canceled)

16. The ADC of claim 1, wherein the ADC has one or more of the following characteristics:
(a) the drug-to-antibody ratio (DAR) is from about 1 to about 4;
(b) the antibody is aglycosylated at N297; and
(c) the antibody comprises an N297Q mutation in the hIgG1 Fc as determined by EU index numbering.

17. (canceled)

18. (canceled)

19. The ADC of claim 1, wherein the antibody or antigen-binding fragment thereof comprises:
an HCDR1 that comprises the amino acid sequence of SEQ ID NO: 4,
an HCDR2 that comprises the amino acid sequence of SEQ ID NO: 6,
an HCDR3 that comprises the amino acid sequence of SEQ ID NO: 8,
an LCDR1 that comprises the amino acid sequence of SEQ ID NO: 12,
an LCDR2 that comprises the amino acid sequence of SEQ ID NO: 14, and
an LCDR3 that comprises the amino acid sequence of SEQ ID NO: 16;
wherein the heavy chain of the antibody or fragment is aglycosylated and comprises an N297Q mutation, and wherein the antibody or fragment is conjugated to tesirine.

20. The ADC of claim 1, wherein the antibody or antigen-binding fragment thereof interacts with at least one residue within the amino acid sequence of SEQ ID NO: 26.

21. The ADC of claim 1, wherein the ADC has one or more of the following characteristics:
(a) demonstrates reduced viability in vivo in EGFRvIII expressing cells;
(b) demonstrates bystander cytotoxicity in vivo against non-EGFRvIII expressing cells co-cultured with EGFRvIII expressing cells;
(c) demonstrates prolonged survival in mice with EGFRvIII expressing intracranial glioblastoma multiforme tumors;
(d) demonstrates anti-tumor effect in mice with EGFRvIII expressing tumors in the absence of treatment related weight loss;
(e) demonstrates tumor regression in mice with patient-derived glioblastoma multiforme tumors;
(f) demonstrates greater tumor killing with lower dosages relative to a comparator antibody conjugated to MMAF; and
(g) demonstrates greater anti-tumor potency than an anti-EGFRvIII-maytansinoid ADC in tumor bearing mice.

22. A complex comprising an ADC of claim 1, wherein the antibody or antigen-binding fragment thereof is bound to EGFRVIII.

23. (canceled)

24. A pharmaceutical composition comprising an ADC of claim 1, and a pharmaceutically acceptable carrier or diluent.

25. (canceled)

26. (canceled)

27. A method for treating a cancer or tumor, or reducing tumor growth, and or causing tumor regression in a subject in need thereof suffering from an EGFRvIII expressing tumor, the method comprising administering to the subject a therapeutically effective amount of an ADC of claim 1.

28. (canceled)

29. The method of claim 27, further comprising:
(a) administering one or more additional therapeutic agents selected from the group consisting of a chemotherapeutic agent, an anti-inflammatory agent, and an analgesic; or
(b) administering a second ADC comprising an antibody or antigen-binding fragment thereof and a cytotoxin, wherein the antibody or antigen-binding fragment thereof of the second ADC specifically binds EGFRvIII and also binds the junctional peptide of SEQ ID NO: 23 and/or the peptide of SEQ ID NO: 24;
wherein the ADC is injected into the body of the subject subcutaneously, intravenously, or intramuscularly.

30. (canceled)

31. (canceled)

32. (canceled)

33. A method for making an ADC of claim 1 comprising culturing a host cell comprising a polynucleotide that encodes an immunoglobulin that comprises the HCVR of said ADC and an immunoglobulin that comprises the LCVR of said ADC, in a culture medium, under conditions favorable to expression of the polynucleotide.

34. The method of claim 33 further comprising conjugating tesirine to one or more of the immunoglobulins.

35. (canceled)

36. (canceled)

37. An ADC that is the product of claim 33.


SEQUENCE LISTING (29 sequences)

SEQ ID NO: 1 - DNA, 366 bp, Artificial Sequence
  Synthetic
gaggtgcagctggtggagtctgggggaggcttggtacagcctggggggtccctgagactctcctgtgcagcctctggattccccttcagtagctacgacatgcactgggtccgccaagctacaggaaaaggtctggagtgggtctcagctattggtactgctggtgccacatactatccaggctccgtgaagggccgattcaccatctccagagaaaatgccaagaactccttgtatcttcaaatgaacagcctgagagccggggacacggctgtgtattactgtgcaagaggggattacgtttgggggacttatcgtcccctctttgactactggggccagggaaccctggtcaccgtctcctca

SEQ ID NO: 2 - protein, 122 aa, Artificial Sequence
  Synthetic
EVQLVESGGGLVQPGGSLRLSCAASGFPFSSYDMHWVRQATGKGLEWVSAIGTAGATYYPGSVKGRFTISRENAKNSLYLQMNSLRAGDTAVYYCARGDYVWGTYRPLFDYWGQGTLVTVSS

SEQ ID NO: 3 - DNA, 24 bp, Artificial Sequence
  Synthetic
ggattccccttcagtagctacgac

SEQ ID NO: 4 - protein, 8 aa, Artificial Sequence
  Synthetic
GFPFSSYD

SEQ ID NO: 5 - DNA, 21 bp, Artificial Sequence
  Synthetic
attggtactgctggtgccaca

SEQ ID NO: 6 - protein, 7 aa, Artificial Sequence
  Synthetic
IGTAGAT

SEQ ID NO: 7 - DNA, 48 bp, Artificial Sequence
  Synthetic
gcaagaggggattacgtttgggggacttatcgtcccctctttgactac

SEQ ID NO: 8 - protein, 16 aa, Artificial Sequence
  Synthetic
ARGDYVWGTYRPLFDY

SEQ ID NO: 9 - DNA, 321 bp, Artificial Sequence
  Synthetic
gacatccagttgacccagtctccatccttcctgtctgcatctgtaggagacagagtcaccatcacttgctgggccagtcagggcattaacaattatttagcctggtatcaacaaaaaccagggaaagcccctaagctcctgatctatgctgcatccactttgcaaactggggtcccatcaaggttcagcggcagtggatctgggacagaattcactctcacaatcagcagcctgcagcctgaagattttgcaacttattactgtcagcagcttaatagttacccgctcactttcggcggagggaccaaggtggagatcaaa

SEQ ID NO: 10 - protein, 107 aa, Artificial Sequence
  Synthetic
DIQLTQSPSFLSASVGDRVTITCWASQGINNYLAWYQQKPGKAPKLLIYAASTLQTGVPSRFSGSGSGTEFTLTISSLQPEDFATYYCQQLNSYPLTFGGGTKVEIK

SEQ ID NO: 11 - DNA, 18 bp, Artificial Sequence
  Synthetic
cagggcattaacaattat

SEQ ID NO: 12 - protein, 6 aa, Artificial Sequence
  Synthetic
QGINNY

SEQ ID NO: 13 - DNA, 9 bp, Artificial Sequence
  Synthetic
gctgcatcc

SEQ ID NO: 14 - protein, 3 aa, Artificial Sequence
  Synthetic
AAS

SEQ ID NO: 15 - DNA, 27 bp, Artificial Sequence
  Synthetic
cagcagcttaatagttacccgctcact

SEQ ID NO: 16 - protein, 9 aa, Artificial Sequence
  Synthetic
QQLNSYPLT

SEQ ID NO: 17 - DNA, 1359 bp, Artificial Sequence
  Synthetic
gaggtgcagctggtggagtctgggggaggcttggtacagcctggggggtccctgagactctcctgtgcagcctctggattccccttcagtagctacgacatgcactgggtccgccaagctacaggaaaaggtctggagtgggtctcagctattggtactgctggtgccacatactatccaggctccgtgaagggccgattcaccatctccagagaaaatgccaagaactccttgtatcttcaaatgaacagcctgagagccggggacacggctgtgtattactgtgcaagaggggattacgtttgggggacttatcgtcccctctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 18 - protein, 452 aa, Artificial Sequence
  Synthetic
EVQLVESGGGLVQPGGSLRLSCAASGFPFSSYDMHWVRQATGKGLEWVSAIGTAGATYYPGSVKGRFTISRENAKNSLYLQMNSLRAGDTAVYYCARGDYVWGTYRPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1359 bp, Artificial Sequence
  Synthetic
gaggtgcagctggtggagtctgggggaggcttggtacagcctggggggtccctgagactctcctgtgcagcctctggattccccttcagtagctacgacatgcactgggtccgccaagctacaggaaaaggtctggagtgggtctcagctattggtactgctggtgccacatactatccaggctccgtgaagggccgattcaccatctccagagaaaatgccaagaactccttgtatcttcaaatgaacagcctgagagccggggacacggctgtgtattactgtgcaagaggggattacgtttgggggacttatcgtcccctctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccaaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 20 - protein, 452 aa, Artificial Sequence
  Synthetic
EVQLVESGGGLVQPGGSLRLSCAASGFPFSSYDMHWVRQATGKGLEWVSAIGTAGATYYPGSVKGRFTISRENAKNSLYLQMNSLRAGDTAVYYCARGDYVWGTYRPLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 645 bp, Artificial Sequence
  Synthetic
gacatccagttgacccagtctccatccttcctgtctgcatctgtaggagacagagtcaccatcacttgctgggccagtcagggcattaacaattatttagcctggtatcaacaaaaaccagggaaagcccctaagctcctgatctatgctgcatccactttgcaaactggggtcccatcaaggttcagcggcagtggatctgggacagaattcactctcacaatcagcagcctgcagcctgaagattttgcaacttattactgtcagcagcttaatagttacccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 22 - protein, 214 aa, Artificial Sequence
  Synthetic
DIQLTQSPSFLSASVGDRVTITCWASQGINNYLAWYQQKPGKAPKLLIYAASTLQTGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 13 aa, Artificial Sequence
  Synthetic
LEEKKGNYVVTDH

SEQ ID NO: 24 - protein, 16 aa, Artificial Sequence
  Synthetic
CGADSYEMEEDGVRKC

SEQ ID NO: 25 - protein, 356 aa, Artificial Sequence
  Synthetic
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIA

SEQ ID NO: 26 - protein, 19 aa, Artificial Sequence
  Synthetic
GPCRKVCNGIGIGEFKDSL

SEQ ID NO: 27 - protein, 1210 aa, Artificial Sequence [sequence omitted]

SEQ ID NO: 28 - protein, 943 aa, Artificial Sequence
  Synthetic
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 29 - protein, 384 aa, Artificial Sequence
  Synthetic
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIAEQKLISEEDLGGEQKLISEEDLHHHHHH